(19) United States
(12) Patent Application Publication
(10) Pub. No.: US 2022/0008451 A1
(43) Pub. Date: Jan. 13, 2022

(54) FLAVIN ADENINE DINUCLEOTIDE (FAD) FOR USE IN THE PREVENTION AND/OR TREATMENT OF CANCER

(71) Applicant: BIO EVEN, PARIS (FR)

(72) Inventors: Didier PALENI, MATHAY (FR); Jolanda SPADAVECCHIA, Paris (FR)

(21) Appl. No.: 17/289,221

(22) PCT Filed: Oct. 31, 2019
(86) PCT No.: PCT/EP2019/079693

§ 371 Date: Apr. 27, 2021

(30) Foreign Application Priority Data
Oct. 31, 2018 (FR) 1860114

(51) Int. Cl. A61K 31/7084; A61K 47/69; A61K 47/60; A61K 47/52; A61P 35/00

(52) U.S. Cl. CPC A61K 31/7084; A61K 47/6923; A61P 35/00; A61K 47/52; A61K 47/60

(57) ABSTRACT

The invention concerns Flavine adenine dinucleotide (FAD) for use in preventing and/or treating cancer. The FAD is favorably encapsulated at least partially in a particle with a vector to improve its absorption and distribution while limiting its destruction, in particular by blood hydrolases. The invention relates to the pharmaceutical field and more specifically to oncology or cancerology.

### TECHNICAL DOMAIN OF THE INVENTION

The invention relates to Flavin Adenine Dinucleotide (FAD) for use in preventing and/or treating cancer and composition for use in preventing and/or treating cancer comprising FAD associated with a vector.

The invention relates to the pharmaceutical field and more specifically to oncology or cancerology.

### STATE-OF-THE-ART

Cancer is a disease characterized by a cell proliferation which is abnormally large within normal body tissue, such that the survival of the body is threatened. These cells all derive from the same clone, cancer-initiating cell that has acquired certain characteristics enabling it to divide indefinitely.

As the disease evolves, some cells may migrate from their place of production and form metastases.

“Cancer” is a general term for a disease in which certain cells in the human body divide in an uncontrolled manner and are considered to have lost their ability to differentiate. The resulting new cells may form a malignant tumour (a neoplasm) and/or spread throughout the body.

“Cancer” is not only seen in humans. It is a pathology that affects many organisms, even if the frequency of occurrence is reduced compared to humans, for example in sharks.

The aging of the population and the improvement in diagnostics show an increase in the incidence of cancer. Cancer treatment has thus become a public health issue. This treatment must be effective in detecting cancerous cells while preserving the body's healthy cells as much as possible. However, the majority of treatments currently available are increasingly effective by being very aggressive towards cancer cells, but also towards healthy cells leading to undesirable effects that can be particularly disabling.

One purpose of the present invention is to propose a new anti-cancer treatment that is both effective on anti-cancer cells while being as targeted and as non-toxic as possible.

The current invention relates, according to one aspect, to an anti-cancer treatment without toxicity for non-cancerous cells, used alone, without other anti-cancer treatment, or used before conventional anti-cancer treatment in a healthy individual or an individual already suffering from cancer.

### SUMMARY OF THE INVENTION

To achieve this objective, according to an embodiment, the present invention concerns Flavin Adenine Dinucleotide (FAD) for its use in the prevention and/or treatment of cancer.

In general, the invention relates to:

1. Flavin Adenine Dinucleotide (FAD) for use in preventing and/or treating cancer.

FAD for its use in preventing and/or treating cancer as the main active ingredient.

FAD for its use in preventing and/or treating cancer as an adjuvant or neoadjuvant to anticancer treatment.

FAD for its use according to any one of the foregoing embodiments in which the cancer is selected from the group consisting of breast cancer, prostate cancer, lung cancer, airway, upper and/or lower digestive tract cancer, organs of digestion cancer, kidney cancer, urinary tract cancer, genital organs cancer, skin cancer, ear-nose-throat (ENT) sphere cancer, and lymphatic organs cancer.

The invention relates to

a composition comprising FAD in a therapeutically effective amount and a pharmaceutically acceptable carrier, preferably for use in the prevention and/or treatment of cancer.

According to an embodiment, the invention relates to the composition according to the preceding embodiments for its use in the prevention and/or treatment of cancer comprising a particle comprising a vector and FAD at least partially encapsulated by the vector.

The composition for its use according to any of the above embodiments in which the vector is chosen from one of at least one of metal nanoparticles, including gold nanoparticles, biopolymers including Poly Ethylene Glycol (PEG), chitosan, collagen, glucose.

The composition for its use according to the above embodiments in which the particle is a nanoparticle or a microparticle.

The composition for its use in any of the above embodiments in which FAD is bound to a biopolymer and a gold nanoparticle.

The composition for its use according to any of the above embodiments in which FAD is covalently bonded to PEG encapsulating at least one gold atom.

The composition for its use according to the above embodiments in which the FAD is bound to gold atoms by coordination binding and bound to the PEG by covalent binding.

The composition for its use according to any one of the above embodiments in which the FAD is at least partially encapsulated by at least one biopolymer preferably selected from PEG, chitosan, glucose.

The composition for its use according to any one of the above embodiments in which the therapeutically effective amount of FAD comprises a quantity of free FAD and a quantity of FAD associated with a vector.

The composition for its use according to any one of the above embodiments in a form suitable for parenteral administration including intravenous, intramuscular, and subcutaneous administration, for vaginal or rectal administration.

The composition for its use according to any one of the above embodiments in a form suitable for intra-vesical or intra-urethral administration.

Unexpectedly, The applicant was able to identify that FAD has interesting properties for use in the prevention and/or treatment of cancer.

The present invention relates to Flavin Adenine Dinucleotide (FAD) as a drug acting alone to treat a disease, and more particularly FAD as a drug acting directly on cancer, or for the treatment of cancer, and/or a cancer-related complication (e.g. angiogenesis, metastasis dissemination).

The present invention relates to the anti-cancer and/or anti-metastatic properties of FAD which is used as a drug in the preventive and/or curative treatment of cancer, in particular breast cancer or cancer of the liver or bladder.

The presence of two nucleic bases in its molecular structure, adenine, and alloxazine, gives it a double binding capacity with DNA and the ability to interfere with the mechanisms controlling gene expression and/or cellular differentiation.

Alloxazine is a “rare” base compared to ATCG.

An increase in the concentration of FAD has been demonstrated in cancer cells. Without being linked to a theory, this concentration can be interpreted as an early attempt at cell repair.

Furthermore, FAD is not toxic to normal blood monocytes PBM cells (GI 50>100 μM).

FAD is advantageously used as the main active ingredient or as an adjuvant or neoadjuvant in cancer treatment.

More advantageously, FAD used as the sole active principle as an anti-cancer treatment is an object of the present invention.

According to another embodiment, FAD is used as the sole active principle as an adjuvant or neoadjuvant for the treatment of cancer.

According to an aspect, FAD associated with a vector (FAD-vector) is an object of the present invention,

FAD-vector is used as the sole active ingredient and/or as an adjuvant or neoadjuvant for the treatment of cancer.

According to another aspect, the present invention relates to a composition comprising FAD, advantageously a therapeutically effective amount of FAD, and a pharmaceutically acceptable carrier or excipient.

According to another aspect, the present invention relates to a composition comprising FAD, advantageously a therapeutically effective amount of FAD, and at least a pharmaceutically acceptable carrier or excipient.

According to another aspect, the present invention relates to a composition comprising a vector and FAD, advantageously a therapeutically effective amount of FAD and a vector, and a pharmaceutically acceptable carrier or excipient.

According to another aspect, the present invention relates to a composition, advantageously a pharmaceutical one, comprising FAD and a biopolymer and/or a metal salt.

According to another aspect, the present invention concerns a composition, advantageously pharmaceutical, comprising a fatty acid.

Hereafter, according to the invention, FAD means FAD alone, and/or FAD associated with at least one vector, preferably FAD associated with at least one vector. FAD according to the invention acts directly on cancerous or precancerous cells.

In the pharmaceutical composition according to the invention, a FAD-vector compound may be present, preferably FAD-vector selected from FAD-polymer, FAD-fatty acid, FAD-biopolymer, or FAD-metal salt, and a combination of these compounds.

A compound comprising a polymer, metal salt, FAD (polymer, metal salt, FAD) is another object of the present invention.

A compound (polymer, metal salt, FAD, fatty acid) is another object of the present invention.

Advantageously the FAD is brought into the presence of fatty acid, preferably a fatty acid that can form a liposome or a capsule comprising encapsulated FAD.

The following embodiments (FAD according to the invention) are part of the invention:

FAD, (FAD, polymer), (FAD, metal salt), (FAD, biopolymer), (FAD, polymer, metal salt), (FAD, bioPolymer, metal salt), (FAD, fatty acid), (FAD, polymer, fatty acid), (FAD, metal salt, fatty acid), (FAD, biopolymer, fatty acid), (FAD, polymer, metal salt, fatty acid), (FAD, biopolymer, metal salt, fatty acid), (FAD, biopolymer, metal salt, amphiphilic fatty acid).

Advantageously, FAD alone or in combination with a metal salt and/or a polymer according to the invention may be combined with a fatty acid to form a vesicle or capsule, such as a liposome, comprising a proportion of encapsulated FAD.

Advantageously, FAD alone (FAD), (FAD, polymer), (FAD, metal salt), (FAD, biopolymer), (FAD, polymer, metal salt), (FAD, biopolymer, metal salt), (FAD, fatty acid), (FAD, polymer, fatty acid), (FAD), metal salt, fatty acid), (FAD, biopolymer, fatty acid), (FAD, polymer, metal salt, fatty acid), (FAD, biopolymer, metal salt, fatty acid), is in the form of microparticles (MP) or nanoparticles (NP).

Advantageously, FAD alone or in combination with a metal salt and/or a biopolymer according to the invention may be combined with a fatty acid to form a vesicle or capsule, such as a liposome, comprising a proportion of encapsulated FAD.

According to another aspect, the present invention relates to a composition, advantageously pharmaceutical for its use in the prevention and/or treatment of cancer, comprising a therapeutically effective amount of FAD and a pharmaceutically acceptable carrier.

According to another aspect, the invention concerns a composition for its use in the prevention and/or treatment of cancer comprising at least one particle comprising FAD according to the invention, in which the FAD is at least partially encapsulated by a vector. Advantageously, the vector has a protective role for the FAD limiting its degradation by enzymes.

According to another aspect, the invention concerns a composition for its use in the prevention and/or treatment of cancer comprising a particle comprising a vector and FAD at least partially encapsulated by the vector. Advantageously, the vector has a protective role for the FAD limiting its degradation by enzymes.

The particle makes it possible to improve the absorption and distribution of FAD in the body while limiting its degradation, particularly by blood hydrolases.

According to another aspect, the present invention concerns a composition, advantageously pharmaceutical, comprising an amount of FAD therapeutically effective on cancer, more particularly consisting of an amount of FAD therapeutically effective on cancer, associated with a biopolymer and/or a metal salt, in the form of a particle and an excipient.

FAD according to the invention means any one of the embodiments described in this description comprising FAD. The FAD may be free and/or associated with a vector, preferably the FAD associated with at least one vector, more preferably the FAD associated with at least one vector selected from a metal salt, a biopolymer, a fatty acid, a combination of vectors.

The invention, therefore, relates to:

FAD as the sole anti-cancer agent, for its use in the prevention and/or treatment of cancer, or as an adjuvant or neoadjuvant of anti-cancer treatment.

FAD as an anti-metastatic agent.

FAD as an anti-precancer agent, or as an anti-cancer agent, FAD according to the invention also covers,

FAD and a chemotherapeutic agent, preferably an antimetabolite and more preferably 5-FU.

A compound or particle comprising FAD and at least one vector, A vesicle, a capsule, a particle comprising any of the FADs according to the invention, preferably a microvesicle, a microcapsule, a microparticle comprising any of the FADs according to the invention, preferably a nanovesicle, a nanocapsule, a nanoparticle comprising any of the FADs according to the invention.

A pharmaceutical composition comprising any of the FADs according to the invention and an excipient,

FAD according to the invention,

According to certain aspects of the invention, FAD can be covalently or non-covalently bound to at least one vector: (FAD-vector).

A vector according to the invention may be selected from a metal salt, a polymer, preferably a biopolymer, a lipid, preferably a lipid amphiphilic, and a combination of vectors.

FAD-HAuCl4, FAD-PEG600, FAD-HAuCl4-PEG600, FAD-chitosan, FAD-HAuCl4-chitosan, FAD-human collagen I, FAD-HAuCl4-human collagen I, FAD-alginate, and FAD-HAuCl4-alginate are preferred examples of FAD according to the invention.

FAD and/or FAD according to the invention encapsulated in liposomes, preferably in the form of nanoparticles, are objects of the present invention.

The invention relates to a particle comprising FAD, preferably a microparticle of FAD (MP) according to the invention, and more preferably a nanoparticle of FAD (NP) according to the invention.

The invention relates to a particle (comprising the FAD and a vector according to the invention) which may also be in the form of a vesicle or capsule, microvesicle or microcapsule, nanovesicle or nanocapsule in which the FAD is encapsulated.

The metals of the salts of metals combined with the FAD according to the invention are selected from Au, Cu, Pd, Gd, Er, Mn, Ag, Co, Zn, Fe, and Ti, preferably Au, more preferably HAuCl4*6H20.

Thus, a vector associated with FAD can be selected from an Au salt, a Cu salt, a Pd salt, a Gd salt, an Er salt, an Mn salt, an Ag salt, a Co salt, a Zn salt, a Fe salt, and a Ti salt, a combination of these salts, preferably an Au salt, more preferably a salt comprising HAuCl4*6H20.

The present invention relates to a compound (combination or particle) of FAD-metal salt;

The present invention relates in one aspect to a FAD-metal salt combination and a device capable of providing radiation, preferably infrared IR more preferably near infrared IR.

The present invention relates in one aspect to a FAD-metal salt combination and a device capable of providing radiation, preferably a FAD-gold salt (FAD-AU salt) or FAD-gold particle combination and a device capable of providing infrared.

The present invention relates in one aspect to a FAD-metal salt combination and a device capable of providing radiation preferably a FAD-gold salt (FAD-au salt) or FAD-gold particle combination and a device capable of providing infrared.

The device capable of providing radiation is preferably capable of providing near IR.

A FAD-gold salt compound (FAD-AU salt) or FAD-gold particle for the treatment of cancer is an object of the present invention.

A combination—FAD-gold salt (FAD-AU salt) or FAD-gold particle and near IR irradiation, for the treatment of cancer is an object of the present invention.

The vector associated with the FAD may be at least one polymer, preferably at least one biopolymer and more particularly, at least one biopolymer selected from among polyethylene glycol (PEG), PEG-diacid, PEG600-diacid; alginic acid or alginate; Poly-Lactide; Bis-Phosphonate; Gelatin; Maltodextrin; Poly amino acids (Poly-L-lysine, Poly-L-ornithine, Poly-L-arginine); lauryl-polyglucose; chitosan; elastin; hyaluronic acid; cellulose; glucose polymer; and a combination of biopolymers; preferably PEG600-diacid, alginic acid, collagen, chitozan, a glucose polymer is a vector of FAD. More preferably PEG600-diacid is associated with FAD.

A glucose polymer means a polymer such as starch, glycogen, sucrose, lactose, a combination of these glucose polymers.

FAD can therefore be associated or combined with any of the glucose polymers selected from starch, glycogen, sucrose, lactose, a combination of several of these glucose polymers.

Among the possible collagens associated with FAD, collagen I or IV is preferred, even more, preferred human or rabbit collagen I or IV, even more, preferred human collagen I or IV, and even more preferred human collagen I.

A FAD liposome (comprising FAD alone and/or FAD linked to at least one vector according to the invention) is an article according to the invention.

A compound comprising FAD and a vector, the vector comprising at least one nucleic acid, selected from RNA, protein-bound RNA, single-stranded DNA, double-stranded DNA, protein-bound single-stranded DNA, protein-bound double-stranded DNA is an article according to the invention.

The invention relates to, according to an aspect, a microparticle or a nanoparticle comprising a FAD Liposome according to the invention and a targeting agent, more particularly a FAD nanoparticle according to the invention and a targeting agent selected from HIV TAT-1 protein, Galectin (Gal-1, Gal-2, Gal-3, Gal-4, Gal-5, Gal-6 Gal-7); interleukin (IL-6), superoxide dismutase (SOD, MnSOD, SOD2, SOD4), monoclonal antibody (Kv-11 and Kv11, Anti-EGFR) or monoclonal antibody binding domains; aptamer, nucleic acid, a siRNA; preferably HIV-1 Tat1 peptide.

Targeting agents are agents conferring cellular and/or tissue specificity to FAD vesicles or particles or liposomes according to the invention. The targeting agents according to the invention are selected from a protein (HIV TAT-1 protein, Galectins (Gal-1, Gal-2, Gal-3, Gal-4, Gal-5, Gal-6, Gal-7); interleukin-6 (IL-6), superoxide dismutase (SOD, MnSOD, SOD2, SOD4), a monoclonal antibody (Kv-11 and Kv11, Anti-EGFR); an aptamer, a nucleic acid, a siRNA; the HIV-1 Tat1 peptide is preferred.

The present invention relates in one aspect to a pharmaceutical composition comprising FAD in a therapeutically effective amount and a pharmaceutically acceptable excipient.

The present invention relates, in one aspect, to a pharmaceutical composition comprising FAD alone and/or according to any of the embodiments described herein, in a therapeutically effective quantity and a pharmaceutically acceptable excipient.

The present invention relates, according to one aspect, to a pharmaceutical composition comprising FAD alone and/or according to any of the embodiments of preparation described herein, in a therapeutically effective quantity and a pharmaceutically acceptable excipient for its use in combination with surgery and/or radiotherapy, preferably the present invention relates, according to one aspect, to a pharmaceutical composition comprising FAD according to any of the embodiments of preparation described (the FAD according to the invention) in a therapeutically effective quantity and a pharmaceutically acceptable excipient, in combination with radiotherapy and more preferably radiotherapy producing near IR.

Pharmaceutically acceptable “excipient” or carrier means any compound which facilitates the shaping of the pharmaceutical composition and does not alter the nature of the biological activity of the active ingredient. A pharmaceutically acceptable excipient may be a solvent, plasticizer, lubricant, dispersion medium, absorption retarding agent, flow agent, etc. Preferably, the composition further comprises a plasticizer, lubricant, and/or flow agent.

A pharmaceutical composition according to the invention for use in the prevention and/or treatment of a disease, preferably cancer, is another aspect of the invention.

A composition according to the invention wherein the therapeutically effective amount of FAD comprises an amount of free FAD and an amount of FAD associated with a carrier is preferred.

An aspect of the invention is that a composition according to the invention is formulated in a form suitable for parenteral administration including intravenous, intramuscular, and subcutaneous administration, mucosal administration e.g. oral, intranasal, vaginal, or rectal, is an aspect of the invention.

Other aspects of the invention are a composition according to the invention in a form suitable for intra bladder or intraurethral administration.

According to another aspect, the present invention concerns a pharmaceutical composition or a kit, advantageously pharmaceutical, comprising the FAD alone or the FAD according to the invention in a therapeutically effective quantity, and at least one other medicinal product.

According to another aspect, the present invention concerns a pharmaceutical composition or a kit comprising FAD or FAD according to the invention in a therapeutically effective quantity, and at least one other medicinal product for its use in combination with surgery and/or radiotherapy, or in combination with exeresis and/or rays, preferably near-infrared rays.

According to another aspect, the present invention concerns a pharmaceutical composition or a kit comprising FAD in a therapeutically effective quantity, and at least one medicinal product.

According to another aspect, the present invention relates to a pharmaceutical composition or kit comprising FAD in a therapeutically effective quantity, and at least one drug used in chemotherapy, preferably in anti-cancer chemotherapy.

According to another aspect, the present invention concerns a pharmaceutical composition or kit comprising (a) FAD in a therapeutically effective amount, and (b) at least one drug used in cancer chemotherapy for use in the treatment of cancer.

A drug used in anticancer chemotherapy associated with FAD according to the invention may be selected from a drug used in conventional chemotherapy, a drug used in hormone therapy, a drug used in immunotherapy, a drug used in targeted immunotherapy.

Anti-cancer chemotherapy is a general drug-based treatment aimed at destroying cancer cells or preventing them from multiplying throughout the body. There are many different chemotherapy drugs, often combined together to increase the effectiveness of the treatment.

According on one aspect, chemotherapy can be prescribed in combination with FAD according to on the invention:

In an adjuvant situation, once the tumour has been removed, ensure that no cancer cells remain.

In a neo-adjuvant situation, reducing the size of the tumor before surgery: makes it possible to limit the potential sequels related to surgery.

In case of metastatic disease, to treat the whole body. There are chemotherapies, which are used depending on the characteristics of cancer.

FAD according to the invention is a new drug to prevent or treat metastatic disease.

According to one aspect, the invention relates to FAD according to the invention, used before, during, and/or after i) surgery, preferably an excision, ii) radiotherapy, preferably IR, iii) chemotherapy, preferably anti-cancer chemotherapy, preferably classical (anti-cancer) chemotherapy such as chemotherapy using synthetic or plant-derived products, hormone therapy, immunotherapy, targeted immunotherapy.

An exeresis is a surgical operation consisting of removing from the body an element that is harmful or useless to it (organ, tumour, foreign body, etc.).

Immunotherapy aims to help the immune system recognise and attack pathological cells, particularly cancerous cells; some seek to bypass the mechanisms that prevent T lymphocytes from triggering an immune response, while others seek to stimulate the specific immune response.

Immunotherapy involves treating a patient with an immunity-derived compound, such as an antibody, fusion protein, receptor, preferably an antibody, and more preferably a monoclonal antibody.

Among the chemotherapy drugs that can be associated with FAD according to the invention, a synthetic or plant-derived product, a hormone-based drug, comprising a hormone agonist or antagonist, a drug comprising an immunity-derived protein, such as an antibody, or an immune cell selected or modified to act specifically on a cellular or tissue target.

According to another aspect, the present invention relates to a pharmaceutical composition or kit comprising FAD in a therapeutically effective amount, and at least one antibody, preferably a monoclonal antibody.

The term “monoclonal antibody” as used here includes, but is not limited to, bevacizumab, cetuximab, trastuzumab, Ibritumomab tiuxetan, rituximab and tositumomab, and iodine 131.

Bevacizumab may be administered in its commercial form, e.g., AVASTIN; cetuximab as ERBITUX; trastuzumab as HERCEPTIN; rituximab as MABTHERA; ibritumomab tiuxetan as ZEVULIN; and tositumomab and iodine 131 as BEXXAR.

Monoclonal antibodies that may be combined with FAD according to the invention include Rituximab, ATC Code L01XC02, Trastuzumab (Herceptin®), ATC Code L01XC03, Gemtuzumab ozogamicin, ATC code L01XC05, Alemtuzumab, ATC code L01XC04, Ibritumomab tiuxetan, ATC code V10XX02, Cetuximab, ATC code L01XC06, Bevacizumab, ATC code L01XC07, Nivolumab, Ipilimumab are particularly suitable.

These monoclonal antibodies are used in the treatment of cancer and therefore can be used in combination with FAD according to the invention, at doses lower than those conventionally used.

Hormone therapy is understood to be a therapy based on the administration of a drug, for example, a hormone, steroid antagonist agonist.

According to a preferred aspect, the present invention relates to a pharmaceutical composition or kit comprising (a) FAD in a therapeutically effective amount, and (b) at least one drug used in conventional cancer chemotherapy.

A medicinal product used in conventional anti-cancer chemotherapy means a chemotherapeutic agent or medicinal product such as an anti-metabolite, an anti-tumour antibiotic, an alkylating agent, an intercalating agent (anthracyclines), a microtubule inhibitor, a topoisomerase I or II inhibitor, a spindle poison (taxoid, vinca-alkaloid, an oxazaphosphorin), a proteasome inhibitor, a protein kinase or phosphatase inhibitor.

The following embodiments of implementation are preferred:

FAD or FAD according to the invention as an anti-metastatic agent, as a curative or palliative anti-cancer agent, more preferably as the only anti-cancer agent. Thus, FAD inhibits the cancerous cells to inhibit the cancerous activity (anarchic proliferation, formation of metastases).

The invention also relates to a:

A pharmaceutical kit comprising FAD according to the invention, for its use in the treatment or prevention of breast cancer,

A pharmaceutical kit comprising FAD according to the invention, for its use in the treatment or prevention of bladder cancer,

A pharmaceutical kit comprising FAD according to the invention, for its use in the treatment or prevention of prostate cancer,

A pharmaceutical kit comprising FAD according to the invention, for its use in the treatment or prevention of lung cancer,

A pharmaceutical kit comprising FAD according to the invention, for its use in the treatment or prevention of airway cancer,

A pharmaceutical kit comprising FAD according to the invention, for its use in the treatment or prevention of cancer of the upper digestive tract,

A pharmaceutical kit comprising FAD according to the invention, for its use in the treatment or prevention of cancer of the lower digestive tract,

A pharmaceutical kit comprising FAD according to the invention, for its use in the treatment or prevention of cancer of the digestive organs,

A pharmaceutical kit comprising FAD according to the invention, for its use in the treatment or prevention of kidney cancer,

A pharmaceutical kit comprising FAD according to the invention, for its use in the treatment or prevention of urinary tract cancer,

A pharmaceutical kit comprising FAD according to the invention, for its use in the treatment or prevention of genital cancer,

A pharmaceutical kit comprising FAD according to the invention, for its use in the treatment or prevention of skin cancer,

A pharmaceutical kit comprising FAD according to the invention, for its use in the treatment or prevention of cancer of the ENT sphere,

A pharmaceutical kit comprising FAD according to the invention, for its use in the treatment or prevention of cancer of the lymphatic organs.

A pharmaceutical kit comprising FAD according to the invention, for its use in the treatment or prevention of breast cancer,

A pharmaceutical kit comprising FAD according to the invention, for its use in the treatment or prevention of bladder cancer,

A pharmaceutical kit comprising FAD according to the invention, for its use in the treatment or prevention of prostate cancer,

A pharmaceutical kit comprising FAD according to the invention, for its use in the treatment or prevention of lung cancer,

A pharmaceutical kit comprising FAD according to the invention, for its use in the treatment or prevention of airway cancer,

A pharmaceutical kit comprising FAD according to the invention, for its use in the treatment or prevention of cancer of the upper digestive tract,

A pharmaceutical kit comprising FAD according to the invention, for its use in the treatment or prevention of cancer of the lower digestive tract,

A pharmaceutical kit comprising FAD according to the invention, for its use in the treatment or prevention of cancer of the digestive organs,

A pharmaceutical kit comprising FAD according to the invention, for its use in the treatment or prevention of kidney cancer,

A pharmaceutical kit comprising FAD according to the invention, for its use in the treatment or prevention of urinary tract cancer,

A pharmaceutical kit comprising FAD according to the invention, for its use in the treatment or prevention of cancer of the genital organs,

A pharmaceutical kit comprising FAD according to the invention, for its use in the treatment or prevention of skin cancer,

A pharmaceutical kit comprising FAD according to the invention, for its use in the treatment or prevention of cancer of the ENT sphere,

A pharmaceutical kit comprising FAD according to the invention, for its use in the treatment or prevention of cancer of the lymphatic organs.

FAD according to the invention for use in combination with an infrared (IR) producing device, a device preferably producing near IR is another object of the invention.

Infrared is electromagnetic radiation; the infrared range is divided into near-infrared (0.5 μm to 5 μm, preferably 0.7 μm<λ<3 μm), middle infrared (3μ<A<25 μm m) and far infrared (beyond 25 μm).

Near-infrared (0.5 μm to 5 μm) is preferred, in combination with a metal salt—FAD, especially Au-FAD salt.

FAD according to the invention for its use in combination with a treatment with infrared, preferably near IR, is another subject of the invention.

According to one aspect, the invention relates to a process for manufacturing particles (FAD, vector), preferably microparticles of (FAD, vector), and even more preferably nanoparticles of (FAD, vector), in which a vector chosen from a metal salt, a biopolymer, an amphiphilic lipid is mixed with the FAD.

According to one aspect, the present invention relates to a drug comprising FAD for treating cancer.

In one aspect the present invention relates to a combination of a drug comprising FAD associated with a biopolymer with another anti-cancer drug, more preferably the drug comprising FAD associated with a biopolymer is combined with an anti-metabolite, even more, preferably the drug comprising FAD associated with a biopolymer is combined with an pyrimidine antagonist antimetabolite, and even more preferably the drug comprising FAD associated with a biopolymer is combined with an pyrimidine antagonist antimetabolite, 5FU.

### BRIEF DESCRIPTION OF THE FIGURES

FIG. 1 illustrates the process of synthesis of a gold-FAD-PEG particle by the IN method.

FIG. 2 illustrates the release of FAD from a gold-FAD-PEG particle obtained by the IN method at pH 5 on UV-Visible spectra.

FIG. 3 illustrates the protection of the FAD by different vectors against free FAD on a graph representing the FAD concentration as a function of time.

FIG. 4 illustrates the temperature variation of the cells exposed to particles of the invention by photothermy.

FIG. 5 illustrates a UV-Visible spectrum before and after encapsulation of FAD in micelle by PEG.

FIG. 6 illustrates the particle stability of FAD-PEG at pH 4 on UV-Visible spectra.

FIG. 7 illustrates FAD-PEG particle hydrolysis as a function of time.

### DETAILED DESCRIPTION OF THE INVENTION

Before embarking on a detailed review of the embodiments of the invention, optional features are set out below which may be used in combination or alternatively:
The invention relates advantageously to FAD for its use as the sole active principle for the prevention and/or treatment of cancer.
The invention relates advantageously to FAD for its use as an anti-cancer agent.
The invention relates advantageously to FAD for its use as the main active ingredient.
The invention relates advantageously to FAD for its use as an adjuvant or neoadjuvant of anti-cancer treatment.
The cancer is advantageously selected from the group consisting of cancer of the breast, prostate, lung, airways, upper and/or lower digestive tract, organs of digestion, kidney, urinary tract, genital organs, skin, ENT sphere, and lymphatic organs.
Cancer, advantageously, is breast cancer,
Cancer, advantageously, is prostate cancer,
Cancer, advantageously, is the cancer of the bladder,
Cancer, advantageously, is lung cancer,
Cancer, advantageously, is the cancer of the airways,
Cancer, advantageously, is the cancer of the upper and/or lower digestive tract,
Cancer, advantageously, is the cancer of the digestive organs,
Cancer, advantageously, is kidney cancer,
Cancer, advantageously, is the cancer of the urinary tract,
Cancer, advantageously, is the cancer of the genitals organs,
Cancer, advantageously, is skin cancer,
Cancer, advantageously, is the cancer of the ENT sphere
Cancer, advantageously, is the cancer of the lymphatic organs.
Cancer, advantageously, is the cancer of the ENT sphere and lymphatic organs.
Cancer, advantageously, is breast cancer,
Cancer, advantageously, is hepatic metastasis,
Cancer, advantageously, is liver cancer,
Cancer, advantageously, is bladder cancer.
The invention, advantageously, relates to a particle comprising a vector and FAD, a composition comprising at least one particle comprising a vector and FAD and a pharmaceutically acceptable carrier.
The invention, advantageously, relates to a particle comprising a vector and FAD in an effective amount for the treatment or prevention of cancer, more advantageously a microparticle (MP) comprising a vector and FAD in an effective amount of a nanoparticle (NP) comprising a vector and FAD in an effective amount. MNP refers to micro or nanoparticle.
The invention, advantageously, relates to a particle comprising a biopolymer and FAD in an effective quantity, more advantageously a microparticle comprising a biopolymer and FAD in an effective quantity, or a nanoparticle comprising a vector and FAD in an effective quantity.
The invention, advantageously, concerns a composition for its use in the prevention and/or treatment of cancer comprising a particle comprising a vector and FAD at least partially encapsulated by the vector.
The invention, advantageously, relates to a composition for use in the prevention and/or treatment of cancer comprising a particle comprising a vector and FAD at least partially protected from enzymatic degradation by the vector.

The FAD may, according to the invention, be non-covalently bound, or covalently bound to a vector (metal salt, biopolymer, fatty acid, phospholipids, liposomes), whether encapsulated, partially encapsulated, or non-encapsulated.

When the FAD is non-covalently bound, or covalently bound to the vector, it is partially protected from degradation enzymatic by the vector, (or partially encapsulated, by extension of the concept of encapsulation).

Preferably, the FAD non-covalently bound or covalently bound to the vector according to the invention is partially and temporarily protected from enzymatic degradation by the vector, (or partially encapsulated, by extension of the concept of encapsulation). According to these modes of implementation, the degradation of the FAD is slowed down, and/or its anti-cancer activity is improved because the FAD is concentrated at the site where it is most effective.

Partially encapsulated means that a proportion of the total FAD is protected from enzymatic degradation because it is encapsulated, and/or that the FAD is covalently or non-covalently bound to a vector which, by steric hindrance, protects the FAD.

According to different embodiments of the invention, the quantity of FAD and therefore its therapeutic action on cancer cells can be concentrated, controlled, and targeted (immediate or progressive release of varying concentrations).

Encapsulated means that the FAD is encapsulated in a vesicle or capsule, arranged inside a particle, or covered with a vector (e.g. polymer or biopolymer or lipid (amphiphiles), phospolipids, liposomes, forming a particle).

The FAD used for the treatment of cancer according to the invention may be without any vectors (100% is not encapsulated) or associated with a vector, for example, a metal salt (non-covalent bond) and partially protected from enzymatic degradation, (partially encapsulated without being in capsule form) or encapsulated with phospholipids for example (liposome).

According to the invention, FAD can also be associated with a vector such as a biopolymer and be bound in a proportion covalently to the biopolymer, non-covalently to the biopolymer, and a free proportion (FIG. 1).

The proportion of FAD encapsulated in preparations according to the invention.

Thus, depending on the modes of realization according to the invention, a proportion of FAD ranging from 0% to 99.999% of the total FAD can be encapsulated, preferably in vesicles, such as liposomes.

According to certain modes of realization in which a capsule (vesicle) can be formed (for example in a combination of FAD in a liposome) according to the invention, a proportion of FAD ranging from 0.01% to 99.99% of the total FAD by weight can be encapsulated, preferably more than 99%; 98%, 97%, 96%, 95%, 94%, 93%, 92%, 91%, 90%, 89%, 88%, 87%, 86%, 85%, 84%, 83%, 82%, 81%, 80%, more than 70%, more than 60%, more than 50%, more than 40%, more than 30%, more than 20%, more than 10%, more than 5%, more than 1% of the FAD may be encapsulated in relation to the total FAD; the other percentage of FAD, relative to the total FAD, is partially encapsulated because it is partially protected from degradation by covalent or non-covalent bonding with the vector, or free.

Encapsulated FAD can be covalently bound to the vector and encapsulated, non-covalently bound to the vector and encapsulated, not bound to the vector i.e. free and encapsulated.

The proportion of unencapsulated FAD to total FAD in preparations according to the invention.

A proportion of the FAD ranging from 0% to 100% of the FAD, by weight in relation to the total weight of the FAD, preferably a proportion of 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97% 98% 99% 99.9%, to 99.99% by weight in relation to the total weight of the FAD, may be unencapsulated.

The unencapsulated FAD can be covalently bound to the vector, or non-covalently bound to the vector, or not bound to the vector, i.e. free. The proportion of unencapsulated FAD to total FAD in preparations according to the invention.

In certain modes of implementation, a proportion of FAD by weight in relation to the total weight of FAD is free, depending on the preparations, ranging from up to a proportion of 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34% 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97% 98% 99% 99.9% to 99.99% by weight in relation to the total weight of the FAD. “Unencapsulated” FAD means FAD not linked to a vector, encapsulated or not encapsulated.

In FAD and metal salt (vector) preparations, the FAD is not encapsulated but non-covalently bound to the vector or free.

According to these modes of production, a proportion of the FAD in relation to the total FAD can be non-covalently bound.

A proportion of 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34% 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97% 98% 99% 99.9%, to 99.99% by weight in relation to the total weight of the FAD can be non-covalently bound.

In the case of FAD bonded to a polymer such as PEG, the bond may be covalent between the polymer and the FAD, the FAD is bonded to the carrier and not encapsulated, partially encapsulated, or encapsulated.

A proportion of the FAD ranging from 0.001% to 99.999% of the FAD by weight of the total weight of the FAD may be covalently bound to the vector and not encapsulated and may even be found wholly or partly on the outer part of the particle.

The FAD can be covalently bound to a vector and encapsulated or covalently bound and unencapsulated.

A proportion of 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34% 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97% 98% 99% 99.9%, 99.99% by weight in relation to the total weight of the ADF can be covalently bound to a vector.

The encapsulated FAD is protected from enzymatic degradation. The unencapsulated FAD, or FAD non-covalently bound to a vector (example a metal salt), can then be encapsulated. According to these modes of implementation, FAD is available more quickly than covalently bonded and encapsulated FAD.

The proportion of non-covalently bound FAD in relation to the quantity of FAD non-covalently bound to the vector and encapsulated represents 1%, 5%, 10%, 15% 20% 25% 30% 35% 40% 45% 50% 55% 60% 65% 70% 75% 80% 85%, 90% 95% of the total FAD by weight.

Thus in a (particle) according to the invention the quantity of FAD covalently bound and the quantity of FAD non-covalently bound and encapsulated corresponds to no more than 99.99, 99%, 98%, 97%, 96%, 95%, 94%, 93%, 92%, 91%, 90%, 89%, 88%, 87%, 86%, 85%, 84%, 83%, 82%, 81%, 80% of the total FAD; not more than 20% corresponds to non-covalently bound and non-encapsulated FAD.

Thus, according to certain embodiments of realization depending on the invention, the quantity of FAD bound in a non-covalent and non-encapsulated manner corresponds to 0.01% to 20% of the total FAD. The proportion of encapsulated FAD (covalently bound or not) and from 99.99% to 80%, the proportion of covalently bound FAD (encapsulated or not) varies according to the quantity of biopolymer and represents between 99% and 1% of the total FAD.
Advantageously, the vector is selected from at least one of the metal nanoparticles, including gold nanoparticles, biopolymers including Poly Ethylene Glycol (PEG), chitosan, collagen, and glucose,
Advantageously, a vector according to the invention is selected from a metal salt, a nanoparticle or a microparticle of metal, more advantageously gold nanoparticles,
A vector according to the invention is selected from, a biopolymer, selected from Poly Ethylene Glycol (PEG), chitosan, collagen, a glucose biopolymer.
A vector according to the invention is chosen from a biopolymer selected from PEG, PEG-diacid, alginic acid, Poly-Lactide, Bis-Phosphonate, Gelatine, Maltodextrin, Poly amino acids (Poly-L-lysine, Poly-L-ornithine, Poly-L-arginine), lauryl-polyglucose, chitosan, collagen, or a combination of these biopolymers, advantageously PEG-diacid and/or chistosan, more advantageously PEG-600 diacid.
Advantageously, the particle is preferably a nanoparticle or a microparticle,
Advantageously, FAD is preferably bound to at least one biopolymer and at least one gold nanoparticle,
Advantageously, FAD is covalently bonded to PEG encapsulating at least one gold nanoparticle,
Advantageously, FAD is bonded to gold nanoparticles by coordination bonding and is covalently bonded to the PEG.
Advantageously, FAD is preferably encapsulated at least partially by at least one biopolymer, preferably selected from PEG, chitosan, glucose, preferably by a PEG micelle,
Advantageously, the particle preferably contains at least one targeting agent on its surface,
Targeting agents aim to make the particle's penetration into cancer cells more specific.
Advantageously, the composition comprises a therapeutically effective amount of FAD, advantageously as the main active ingredient, or as an adjuvant or neoadjuvant of anti-cancer treatment, and a pharmaceutically acceptable carrier.
Advantageously, the therapeutically effective amount of FAD comprises an amount of free FAD and an amount of FAD associated with a vector,
Free FAD is defined as FAD not associated with a vector. This combination of two forms of FAD ensures immediate efficacy from the moment of administration and prolonged efficacy by the FAD associated with the vector, which thus presents bioavailability over a longer period of time.
Advantageously, the composition is preferably formulated for parenteral administration, including intravenous, intramuscular, and subcutaneous administration, and vaginal or rectal administration.
The composition is preferably in a suitable form for intra bladder administration (intravesicale).

According to another aspect, the present invention concerns a process for the synthesis of nanoparticles, by complexation and/or encapsulation with pharmacologically acceptable excipients. In this way, capsules comprising polyethylene glycols (PEG) and targeting agents, or liposomes can be produced. Targeting agents aim to make the particle's penetration into cancer cells more specific.

According to another aspect, the present invention concerns a process for the synthesis of FAD nanoparticles, by FAD complexation and/or encapsulation with pharmacologically acceptable excipients.

In this way, capsules comprising polyethylene glycols (PEG) and targeting agents, or liposomes can be produced. Targeting agents aim to make the particle's penetration into cancer cells more specific.

Flavin Adenine Dinucleotide (FAD) is a redox co-factor made from Flavin Mononucleotide (FMN). It is associated with oxidoreductase enzymes. It is a water-soluble, non-proteinaceous organic molecule.

FAD is a molecule of formula I below:

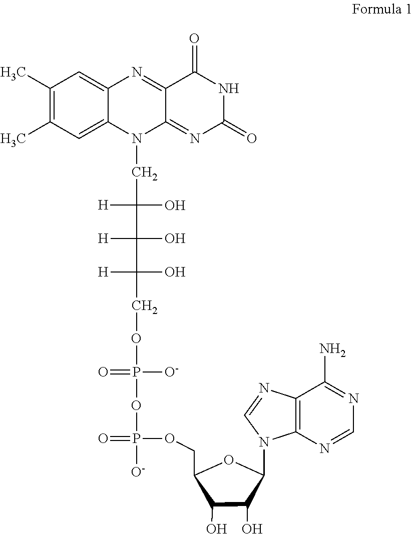

It is commercialised in particular by Alfa Aesar and is registered under the number CAS 146-14-5.

FAD is naturally synthesised by the body and is composed of a vitamin B2-type molecule on the one hand, and an adenine nucleic base is formerly known as vitamin B4 on the other, both of which are perfectly safe.

FAD differs from vitamin B2, from a structural point of view, by the presence of the adenine nucleic base, and from the point of view of their functions and distribution in the human body.

FAD is a co-factor in oxidation-reduction reactions. It accounts for 70 to 90% of the total riboflavin (RT) in the body distributed in cellular tissues. Vitamin B2 represents only 0.5 to 2% of total riboflavin and is mainly involved as a constituent of FMN (5 to 30% of RT). It is present in the urine.

The bioavailability of FAD is limited, however, because the molecule is degraded in the blood as circulating FMN (Flavin Mononucleotide).

FAD plays a role as a coenzyme in many biological reactions in the body. However, to the applicant's knowledge, FAD has never been used for the treatment and/or prevention of cancer.

According to the invention, FAD is used to treat cancers. Without being linked to a theory, it has been found that FAD reduces the viability of cancer cells.

The inventors demonstrate in a surprising way that FAD intercalates in the DNA and can therefore act directly on cancer cells.

According to the invention, FAD is advantageously used to prevent cancers, i.e. FAD plays a preventive role. The preventive treatment of cancer is based on the absence of toxicity of FAD and its capacity to inhibit the growth of cancer cells at an early stage, for example by directing them towards apoptosis or by differentiating them, i.e. by directing their cellular maturation towards their predestination organ.

According to a embodiment of realization of the invention, FAD is used as an active principle on its own. The FAD according to the invention is not only used as a biological potentiator of another anticancer active principle or not.

According to one aspect of the invention, FAD is used as a neoadjuvant. As a neo-adjuvant, it prepares the main treatment for tumour removal surgery. Neo-adjuvant means a treatment designed to reduce the size of the tumour or stabilize it before performing surgery or radiotherapy, which it makes easier.

According to one aspect of the invention, FAD is used as an adjuvant, it complements surgical, chemotherapy, or radiotherapy treatments, preventing the risk of local recurrence or metastasis.

By maintaining an inhibitory pressure on the growth of cancer cells, FAD consolidates the results of chemotherapy in the interval between two treatment sequences. And it reduces the side effects through its metabolic action on healthy cells.

FAD is preferably used for the prevention and/or treatment of cancers selected from solid tumours and lymphomas. The cancer is preferably selected from the group consisting of breast, prostate, lung, airway, upper and/or lower digestive tract, organs of digestion such as stomach, liver, pancreas, kidney, urinary tract, genitals, skin, ENT sphere, and lymphatic organs. It is also used in the treatment of leukaemia.

More preferably, FAD is used for the prevention and/or treatment of breast cancer.

More preferably, FAD is used for the prevention and/or treatment of liver cancer.

More preferably, FAD is used for the prevention and/or treatment of prostate cancer.

More preferably, FAD is used for preventing and/or treating cancer of the airways.

More preferably, FAD is used for preventing and/or treating cancer of the upper digestive tract.

More preferably, FAD is used for preventing and/or treating cancer of the lower digestive tract.

More preferably, FAD is used for preventing and/or treating cancer of the digestive organs.

More preferably, FAD is used for preventing and/or treating stomach cancer.

More preferably, FAD is used for the prevention and/or treatment of pancreatic cancer.

More preferably, FAD is used for the prevention and/or treatment of kidney cancer.

More preferably, FAD is used for preventing and/or treating cancer of the urinary tract.

More preferably, FAD is used for preventing and/or treating cancer of the genital organs.

More preferably, FAD is used for preventing and/or treating skin cancer.

More preferably, FAD is used for preventing and/or treating cancer of the ENT sphere.

More preferably, FAD is used for preventing and/or treating cancer of the lymphatic organs.

It is also used in the treatment of leukaemia.

In a more preferred mode, FAD is used to stabilise liver cancer and/or prevent metastases from forming.

In a more preferred mode, FAD is used to stabilize breast cancer and/or prevent metastases from forming.

In a more preferred mode, FAD is used to stabilize pancreatic cancer and/or prevent metastases from forming.

According to a preferred embodiment, FAD is at least partially encapsulated in a particle to improve its absorption, bioavailability, and/or distribution, advantageously, while limiting its destruction, particularly by pyrophosphatases and/or blood hydrolases. The encapsulated formulation makes it possible to increase the half-life of the FAD.

An at least partially encapsulated FAD means a FAD non-covalently or covalently associated with a vector, advantageously a FAD non-covalently or covalently associated with a biopolymer and/or a metal salt.

Partially encapsulated may mean for the FAD that the vector associated with the FAD partially covers or protects the FAD by steric hindrance. Depending on its association with the different reactive groups of the FAD, such as the NH2 end of the FAD Flavin Mononucleotide (FMN) or for example with the pyrophosphate bond of the FAD AMP, a vector according to the invention (depending on its nature) binds covalently or non-covalently with the FAD.

According to a preferred embodiment, FAD binds non-covalently with a metal salt, preferably a gold salt, to form a particle, preferably a macroparticle, and even more preferably a nanoparticle.

According to the invention, a protective group means a group that protects the FAD from enzymatic degradation. A protective group according to the invention can be a vector according to the invention. Amongst the protective groups of the invention, PEG-600 diacid is preferred.

A protective group according to the invention means a group that protects the FAD from enzymatic degradation for more than 72 hours, more than 48 hours, more than 24 hours, more than 12 hours, more than 8 hours, more than 4 hours, more than 1 hour or which increases the half-life of the FAD by at least a factor of 0.5, 2, 4, 6, 8, 10, 12, 20, 30, 40, 50, 100, 1000. According to the invention, a protective group may be a vector according to the invention selected from a metal salt, a biopolymer, a phospholipid. Amongst the protective groups of the invention, PEG-600 diacid is preferred.

According to different embodiments, the ratio in the number of molecules between the protective group and the FAD is 1:10, 1:2, 1:1, 1:0.5, 1:0.025.

The invention relates preferably to a composition comprising a particle comprising a vector and at least partially encapsulated preferably fully encapsulated FAD.

The term “a particle” is used, but it is understood that the composition comprises a plurality of particles. The particle according to the invention is a microparticle or a nanoparticle.

It is understood in the sense of the invention that the nanoparticles are smaller than 100 nm in size and preferably the nanoparticles according to the invention are smaller than 50 nm in diameter.

For the invention, microparticles are understood to be between 1 and 1000 □m in size, and preferably microparticles according to the invention are less than 100 □m in diameter.

The particles may be capsules, micelles, liposomes in which the FAD is at least partially, preferably surrounded by at least one vector, or spheres in which the vector forms a matrix in which the FAD is dispersed.

A vector according to the invention means a compound or material which covalently and/or non-covalently associated with the FAD may have one of the following effects on the FAD selected from (i) slowing the degradation of FAD, (ii) temporarily protecting the FAD from enzymatic degradation, (iii) concentrating or targeting the FAD at the site of action, create a FAD gradient, allow a prolonged release of the FAD, allow an action such as reacting to infrared irradiation to provoke intracellular hyperthermia that can be used therapeutically, induce an enzymatic reaction, in particular at the level of a nucleic acid or several of these effects.

A vector according to the invention may comprise a metal particle, a metal nanoparticle, a metal salt; a polymer, such as a biopolymer; a lipid, such as a phospholipid; or a combination of these compounds.

According to certain aspects of the invention, the vector comprises a biopolymer or a mixture of biopolymers associated alone with the FAD or associated with the FAD and at least one metal, or a metal salt preferably in nanoparticle form.

According to certain embodiments, a biopolymer according to the invention associated with FAD is a biocompatible polymer selected from the group consisting of chitosan, elastin, hyaluronic acid, alginate, gelatin, collagen, cellulose, glucose, and polyethylene glycol (PEG).

According to certain embodiments, a biopolymer according to the invention associated with FAD is a biocompatible polymer selected from the group consisting of chitosan, elastin, hyaluronic acid, alginate, gelatin, collagen, cellulose, a glucose polymer, and polyethylene glycol (PEG).

According to certain embodiments, a biopolymer is a biocompatible polymer associated with FAD selected from the group consisting of chitosan, elastin, hyaluronic acid, alginate, gelatin, collagen, cellulose, glucose polymer, polyethylene glycol (PEG), PEG-Diacid (PEG 600 diacid), Alginic acid (ALG or alginate according to PH), Poly-Lactide, Bis-Phosphonate, Gelatine, Maltodextrin, Polyamino acids (Poly-L-lysine, Poly-L-ornithine, Poly-L-arginine, lauryl-polyglucose, chitosan, collagen (Collagen I or IV).

According to certain aspects, the biopolymers according to the invention associated with FAD are selected from PEG-Diacid (PEG 600 diacid), alginic acid (ALG or alginate), Poly-Lactide, Bis-Phosphonate, Gelatine, Maltodextrin, Poly amino acids (Poly-L-lysine, Poly-L-ornithine, Poly-L-arginine, lauryl-polyglucose, chitosan, collagen (Collagen 1 or IV).

### OTHER SPECIFIC EMBODIMENTS OR OBJECT ACCORDING TO THE INVENTION

Each of the objects (or embodiment) described is an object according to the invention. Each of the objects described may be in the form of particles, preferably microparticles and more preferably in the form of nanoparticles.

### FAD-Biopolymer

The present invention relates to a compound selected from FAD-chitosan, FAD-elastin, FAD-hyaluronic acid, FAD-alginate, FAD-gelatin, FAD-collagen, FAD-cellulose, FAD-glucose polymer, and FAD-polyethylene glycol (PEG).

The present invention relates to a compound selected from a biopolymer-FAD FAD-chitosan, FAD-elastin, FAD-hyaluronic acid, FAD-alginate, FAD-gelatin, FAD-collagen, FAD-cellulose, FAD-glucose polymer, FAD-polyethylene glycol (PEG), FAD-PEG-Diacid (FAD-PEG 600 diacid), FAD-alginic acid (or FAD-ALG or FAD-alginate according to PH), FAD-Poly-Lactide, FAD-Bis-Phosphonate, FAD-Gelatin, FAD-Maltodextrin, FAD-Poly amino acids (FAD-Poly-L-lysine, FAD-Poly-L-ornithine, FAD-Poly-L-arginine, FAD-lauryl-polyglucose, FAD-chitosan, FAD-collagen (FAD-Collagen-I or FAD-collagen-IV).

The FAD-biopolymers according to the invention are selected from FAD-PEG-Diacid (FAD-PEG 600 diacid), FAD-alginic acid (FAD-ALG or FAD-alginate), FAD-Poly-Lactide, FAD-Phosphonate, FAD-Gelatin, FAD-Maltodextrin, FAD-Polyamino acids (FAD-Poly-L-lysine, FAD-Poly-L-ornithine), FAD-Poly-L-arginine, FAD-lauryl-polyglucose, FAD-chitosan, FAD-collagen (FAD-Collagen-I or FAD-Collagen IV), preferably FAD-PEG 600 diacid, FAD-ALG, FAD-chitosan, FAD-Collagen I or FAD-collagen IV, even more preferably FAD-PEG 600 diacid.

### FAD Metals

The metals of salts of metals combined with FAD according to the invention are selected from Au, Cu, Pd, Gd, Er, Mn, Ag, Co, Zn, Fe, Ti. Thus, the FAD can be combined with each of these metal salts to form a compound: Au-FAD salt, Cu-FAD salt, Pd-FAD salt, Gd-FAD salt, ER-FAD salt, Mn-FAD salt, Ag-FAD salt, Co-FAD salt, Zn-FAD salt, Fe-FAD salt, Ti-FAD salt, preferably an Au-FAD salt, more preferably HAuCl4*6H2O-FAD.

Compounds corresponding to FAD-Au salt, FAD-Cu salt, FAD-Pd salt, FAD-Gd salt, FAD-Er salt, FAD-Mn salt, FAD-Ag salt, FAD-Co salt, FAD-Zn salt, FAD-Fe salt, FAD-Ti salt, preferably FAD-Au salt, and more preferably FAD-HAuCl4*6H2O are also part of the invention.

Each of these metal salts individually associated with the FAD can also be bonded to a polymer, preferably a biopolymer according to the invention.

According to the invention, a Polymer-Salt of Au-FAD, Polymer-Salt of Cu-FAD, Polymer-Salt of Pd-FAD, Polymer-Salt of Gd-FAD, Polymer-Salt of Er-FAD, Polymer-Salt of Mn-FAD, Polymer-Salt of Ag-FAD, Polymer-Salt of Co-FAD, Polymer-Salt of Zn-FAD, Polymer-Salt of Fe-FAD, Polymer-Salt of Ti-FAD is manufactured; preferably, a Polymer-Salt of Au-FAD, and more preferably, a Polymer-Salt of HAuCl4*6H2O-FAD.

Compounds corresponding to Polymer-FAD-Au Salt, Polymer-FAD-Cu Salt, Polymer-FAD-Pd Salt, Polymer-FAD-Gd Salt, Polymer-FAD-Er Salt, Polymer-FAD-Mn Salt, Polymer-FAD-Ag Salt, Polymer-FAD-Co Salt, Polymer-FAD-Zn Salt, Polymer-FAD-Fe Salt, Polymer-FAD-Ti Salt are also part of the invention. It is preferable to use Polymer-FAD-Au Salt, and even more Polymer-FAD-HAuCl4*6H2O Salt.

According to the invention, Au-Polymer-FAD Salt, Cu-Polymer-FAD Salt, Pd-Polymer-FAD Salt, Gd-Polymer-FAD Salt, Er-Polymer-FAD Salt, Mn-Polymer-FAD Salt, Ag-Polymer-FAD Salt, Co-Polymer-FAD Salt, Zn-Polymer-FAD Salt, Fe-Polymer-FAD Salt, Ti-Polymer-FAD Salt are manufactured. Au-Polymer-FAD Salt is preferred, and HAuCl4*6H2O-Polymer-FAD salt.

Compounds corresponding to FAD-Polymer-Au Salt, FAD-Polymer-Cu Salt, FAD-Polymer-Pd Salt, FAD-Polymer-Gd Salt, FAD-Polymer-Er Salt, FAD-Polymer-Mn Salt, FAD-Polymer-Ag Salt, FAD-Polymer-Co Salt, FAD-Polymer-Zn Salt, FAD-Polymer-Fe Salt, FAD-Polymer-Ti Salt are also part of the invention, preferably FAD-Polymer-Salt of Au and even more preferably FAD-Polymer-Salt of HAuCl4*6H2O.

### Biopolymers

According to the invention, a BioPolymer-Salt of Au-FAD, BioPolymer-Salt of Cu-FAD, BioPolymer-Salt of Pd-FAD, BioPolymer-Salt of Gd-FAD, BioPolymer-Salt of Er-FAD, Biopolymer-Salt of Mn-FAD, Biopolymer-Salt of Ag-FAD, Biopolymer-Salt of Co-FAD, Biopolymer-Salt of Zn-FAD, Biopolymer-Salt of Fe-FAD, Biopolymer-Salt of Ti-FAD, is manufactured. Preferably BioPolymer-Salt of Au-FAD, and even more HAuCl4*6H2O-FAD Biopolymer-Salt.

Compounds corresponding to Biopolymer-FAD-Au Salt, Biopolymer-FAD-Cu Salt, Biopolymer-FAD-Pd Salt, Biopolymer-FAD-Gd Salt, Biopolymer-FAD-Er Salt, Biopolymer-FAD-Mn Salt, Biopolymer-FAD-Ag Salt, Biopolymer-FAD-Co Salt, Biopolymer-FAD-Zn Salt, Biopolymer-FAD-Fe Salt, Biopolymer-FAD-Ti Salt are also part of the invention. It is preferable to use Biopolymer-FAD-Au Salt and even more Biopolymer-FAD-HAuCl4*6H2O Salt.

According to the invention, an Au Salt-Biopolymer-FAD, Cu Salt-Biopolymer-FAD, Pd Salt-Biopolymer-FAD, Gd Salt-Biopolymer-FAD, ER Salt-Biopolymer-FAD, Mn Salt-Biopolymer-FAD, Ag Salt-Biopolymer-FAD, Co Salt-Biopolymer-FAD, Zn Salt-Biopolymer-FAD, Fe Salt-Biopolymer-FAD, Ti Salt-Biopolymer-FAD, is manufactured. The Au Salt-Biopolymer-FAD and even more HAuCl4*6H2O Salt-Biopolymer-FAD is preferred.

Compounds corresponding to FAD-Biopolymer-Au Salt, FAD-Biopolymer-Cu Salt, FAD-Biopolymer-Pd Salt, FAD-Biopolymer-Gd Salt, FAD-Biopolymer-Er Salt, FAD-Biopolymer-Mn Salt, FAD-Biopolymer-Ag Salt, FAD-Biopolymer-Co Salt, FAD-Biopolymer-Zn Salt, FAD-Biopolymer-Fe Salt, FAD-Biopolymer-Ti Salt are also part of the invention. FAD-Biopolymer-Au Salt and even more FAD-Biopolymer-HAuCl4*6H2O Salt are preferred.

According to the invention, a PEG-Diacid-Salt of Au-FAD, PEG-Diacid-Salt of Cu-FAD, PEG-Diacid-Salt of Pd-FAD, PEG-Diacid-Salt of Gd-FAD, PEG-Diacid-Salt of Er-FAD, PEG-Diacid-Salt of Mn-FAD, PEG-Diacid-Salt of Ag-FAD, PEG-Diacid-Salt of Co-FAD, PEG-Diacid-Salt of Zn-FAD, PEG-Diacid-Salt of Fe-FAD, PEG-Diacid-Salt of Ti-FAD, is manufactured. PEG-Diacid-Salt of Au-FAD is preferred, and even more PEG-Diacid-Salt of HAuCl4*6H2O.

Compounds corresponding to PEG-Diacid-FAD-Au Salt, PEG-Diacid-FAD-Cu Salt, PEG-Diacid-FAD-Pd Salt, PEG-Diacid-FAD-Gd Salt, PEG-Diacid-FAD-Er Salt, PEG-Diacid-FAD-Mn Salt, PEG-Diacid-FAD Ag Salt, PEG-Diacid-FAD Co Salt, PEG-Diacid-FAD Zn Salt, PEG-Diacid-FAD Fe Salt, PEG-Diacid-FAD Ti Salt are also part of the invention. PEG-Diacid-FAD-Au Salt and even more PEG-Diacid-FAD-HAuCl4*6H2O Salt are preferred.

According to the invention, a Salt of Au-PEG-Diacid-FAD, Salt of Cu-PEG-Diacid-FAD, Salt of Pd-PEG-Diacid-FAD, Salt of Gd-PEG-Diacid-FAD, Salt of Er-PEG-Diacid-FAD, Salt of Mn-PEG-Diacid-FAD, Salt of Ag-PEG-Diacid-FAD, Salt of Co-PEG-Diacid-FAD, Salt of Zn-PEG-Diacid-FAD, Salt of Fe-PEG-Diacid-FAD, Salt of Ti-PEG-Diacid-FAD is manufactured. Salt of Au-PEG-Diacid-FAD and even more Salt of HAuCl4*6H2O-PEG-Diacid-FAD are preferred.

Compounds corresponding to FAD-PEG-Diacid-Au Salt, FAD-PEG-Diacid-Cu Salt, FAD-PEG-Diacid-Pd Salt, FAD-PEG-Diacid-Gd Salt, FAD-PEG-Diacid-Er Salt, FAD-PEG-Diacid-Mn Salt, FAD-PEG-Diacid-Ag Salt, FAD-PEG-Diacid-Co Salt, FAD-PEG-Diacid-Zn Salt, FAD-PEG-Diacid-Fe Salt, FAD-PEG-Diacid-Ti Salt are also part of the invention. FAD-PEG-Diacid-Au Salt and even more FAD-PEG-Diacid-HAuCl4*6H2O Au Salt are preferred.

Corresponding to an even more advantageous embodiments PEG-600 diacid-Salt of Au-FAD, PEG-600 diacid-Salt of Cu-FAD, PEG-600 diacid-Salt of Pd-FAD, PEG-600 diacid-Salt of Gd-FAD, PEG-600 diacid-Salt of Er-FAD, PEG-600 diacid-Salt of Mn-FAD, PEG-600 diacid-Salt of Ag-FAD, PEG-600 diacid-Salt of Co-FAD, PEG-600 diacid-Salt of Zn-FAD, PEG-600 diacid-Salt of Fe-FAD, PEG-600 diacid-Salt of Ti-FAD, is manufactured. PEG-600 diacid-Salt of Au-FAD and even more PEG-600 diacid-Salt of HAuCl4*6H2O-FAD is preferred.

Compounds corresponding to PEG-600 diacid-FAD-Salt of Au, PEG-600 diacid-FAD-Salt of Cu, PEG-600 diacid-FAD-Salt of Pd, PEG-600 diacid-FAD-Salt of Gd, PEG-600 diacid-FAD-Salt of Er, PEG-600 diacid-FAD-Salt of Mn, PEG-600 diacid-FAD-Salt of Ag, PEG-600 diacid-FAD-Salt of Co, PEG-600 diacid-FAD-Salt of Zn, PEG-600 diacid-FAD-Salt of Fe, PEG-600 diacid-FAD-Salt of Ti are also part of the invention. PEG-600 diacid-FAD-Salt of Au, and even more PEG-600 diacid-FAD-Salt of HAuCl4*6H2O is preferred.

According to the invention, Au Salt-PEG-600 diacid-FAD, Cu Salt-PEG-600 diacid-FAD, Pd Salt-PEG-600 diacid-FAD, Gd Salt-PEG-600 diacid-FAD, Er Salt-PEG-600 diacid-FAD, Mn Salt-PEG-600 diacid-FAD, Ag Salt-PEG-600 diacid-FAD, Co Salt-PEG-600 diacid-FAD, Zn Salt-PEG-600 diacid-FAD, Fe Salt-PEG-600 diacid-FAD, Ti Salt-PEG-600 diacid-FAD, is manufactured. Au Salt-PEG-600 diacid-FAD, and even more HAuCl4*6H2O Salt-PEG-600 diacid-FAD is preferred.

Compounds corresponding to FAD-PEG-600 diacid-Au Salt, FAD-PEG-600 diacid-Cu Salt, FAD-PEG-600 diacid-Pd Salt, FAD-PEG-600 diacid-Gd Salt, FAD-PEG-600 diacid-Er Salt, FAD-PEG-600 diacid-Mn Salt, FAD-PEG-600 diacid-Ag Salt, FAD-PEG-600 diacid-Co Salt, FAD-PEG-600 diacid-Zn Salt, FAD-PEG-600 diacid-Fe Salt, FAD-PEG-600 diacid-Ti Salt are also part of the invention. FAD-PEG-600 diacid-Au Salt, and even more FAD-PEG-600 diacid-HAuCl4*6H2O Salt is preferred.

### ALG FAD

According to the invention, an ALG-Salt of Au-FAD, ALG-Salt of Cu-FAD, ALG-Salt of Pd-FAD, ALG-Salt of Gd-FAD, ALG-Salt of Er-FAD, ALG-Salt of Mn-FAD, ALG-Salt of Ag-FAD, ALG-Salt of Co-FAD, ALG-Salt of Zn-FAD, ALG-Salt of Fe-FAD, ALG-Salt of Ti-FAD, is manufactured. ALG-Salt of Au-FAD, and even more ALG-Salt of HAuCl4*6H2O-FAD is preferred.

Compounds corresponding to ALG-FAD-Au Salt, ALG-FAD-Cu Salt, ALG-FAD-Pd Salt, ALG-FAD-Gd Salt, ALG-FAD-Er Salt, ALG-FAD-Mn Salt, ALG-FAD-Ag Salt, ALG-FAD-Co Salt, ALG-FAD-Zn Salt, ALG-FAD-Fe Salt, ALG-FAD-Ti Salt are also part of the invention. ALG-FAD-Au Salt and even more ALG-FAD-HAuCl4*6H2O Salt is preferred.

According to the invention, an Au Salt-ALG-FAD, Cu Salt-ALG-FAD, Pd Salt-ALG-FAD, Gd Salt-ALG-FAD, Er Salt-ALG-FAD, Mn Salt-ALG-FAD, Ag Salt-ALG-FAD, Co Salt-ALG-FAD, Zn Salt-ALG-FAD, Fe Salt-ALG-FAD, Ti Salt-ALG-FAD, is manufactured. Au Salt-FAD-ALG and even more HAuCl4*6H2O Salt-FAD-ALG is preferred.

Compounds corresponding to FAD-ALG-Au Salt, FAD-ALG-Cu Salt, FAD-ALG-Pd Salt, FAD-ALG-Gd Salt, FAD-ALG-Er Salt, FAD-ALG-Mn Salt, FAD-ALG-Ag Salt, FAD-ALG-Co Salt, FAD-ALG-Zn Salt, FAD-ALG-Fe Salt, FAD-ALG-Ti Salt are also part of the invention and manufacturing. FAD-ALG-Au Salt, and even more FAD-ALG-HAuCl4*6H2O-FAD Salt is preferred.

CHITOSAN

Chitosan-Au Salt-FAD, Chitosan-Cu Salt-FAD, Chitosan-Pd Salt-FAD, Chitosan-Gd Salt-FAD, Chitosan-Er Salt-FAD, Chitosan-Mn Salt-FAD, Chitosan-Ag Salt-FAD, Chitosan-Co Salt-FAD, Chitosan-Zn Salt-FAD, Chitosan-Fe Salt-FAD, Chitosan-Ti Salt-FAD is manufactured. Chitosan-Au Salt-FAD, and even more Chitosan-HAuCl4*6H2O Salt-FAD is preferred.

Compounds corresponding to Chitosan-FAD-Au Salt, Chitosan-FAD-Cu Salt, Chitosan-FAD-Pd Salt, Chitosan-FAD-Gd Salt, Chitosan-FAD-Er Salt, Chitosan-FAD-Mn Salt, Chitosan-FAD-Ag Salt, Chitosan-FAD-Co Salt, Chitosan-FAD-Zn Salt, Chitosan-FAD-Fe Salt, Chitosan-FAD-Ti Salt are also part of the invention and manufactured. Chitosan-FAD-Au Salt, and even more Chitosan-FAD-HAuCl4*6H2O Salt is preferred.

Au Salt-Chitosan-FAD, Cu Salt-Chitosan-FAD, Pd Salt-Chitosan-FAD, Gd Salt-Chitosan-FAD, Er Salt-Chitosan-FAD, Mn Salt-Chitosan-FAD, Ag Salt-Chitosan-FAD, Co Salt-Chitosan-FAD, Zn Salt-Chitosan-FAD, Fe Salt-Chitosan-FAD, Ti Salt-Chitosan-FAD is manufactured. Au Salt-Chitosan-FAD, and even more HAuCl4*6H2O Salt-Chitosan-FAD is preferred.

Compounds corresponding to FAD-CHITOSAN-Au Salt, FAD-CHITOSAN-Cu Salt, FAD-CHITOSAN-Pd Salt, FAD-CHITOSAN-Gd Salt, FAD-CHITOSAN-Er Salt, FAD-CHITOSAN-Mn Salt, FAD-CHITOSAN-Ag Salt, FAD-CHITOSAN-Co Salt, FAD-CHITOSAN-Zn Salt, FAD-CHITOSAN-Fe Salt, FAD-CHITOSAN-Ti Salt are also part of the invention and manufactured. FAD-CHITOSAN-Au Salt, and even more FAD-CHITOSAN-HAuCI4*6H2O Salt of is preferred.

### Collagen 1

Collagen 1-Salt of Au-FAD, Collagen 1-Salt of Cu-FAD, Collagen 1-Salt of Pd-FAD, Collagen 1-Salt of Gd-FAD, Collagen 1-Salt of Er-FAD, Collagen 1-Salt of Mn-FAD, Collagen 1-Salt of Ag-FAD, Collagen 1-Salt of Co-FAD, Collagen 1-Salt of Zn-FAD, Collagen 1-Salt of Fe-FAD, Collagen 1-Salt of Ti-FAD, is manufactured.

Collagen 1-Salt of Au-FAD, and even more Collagen 1-Salt of HAuCl4*6H2O-FAD is preferred.

Compounds corresponding to Collagen I-FAD-Au Salt, Collagen I-FAD-Cu Salt, Collagen I-FAD-Pd Salt, Collagen I-FAD-Gd Salt, Collagen I-FAD-Er Salt, Collagen I-FAD-Mn Salt, Collagen I-FAD-Ag Salt, Collagen I-FAD-Co Salt, Collagen I-FAD-Zn Salt, Collagen I-FAD-Fe Salt, Collagen I-FAD-Ti Salt are also part of the invention and manufacturing. Collagen I-FAD-Au Salt and even more Collagen I-FAD-HAuCl4*6H2O Salt is preferred.

Au Salt-Collagen I-FAD, Cu Salt-Collagen I-FAD, Pd Salt-Collagen I-FAD, Gd Salt-Collagen I-FAD, ER Salt-Collagen I-FAD, Mn Salt-Collagen I-FAD, Ag Salt-Collagen I-FAD, Co Salt-Collagen I-FAD, Zn Salt-Collagen I-FAD, Fe Salt-Collagen I-FAD, Ti Salt-Collagen I-FAD, is manufactured. Au Salt-Collagen I-FAD and even more HAuCl4*6H2O Salt-Collagen I-FAD is preferred.

Compounds corresponding to FAD-Collagen I-Au Salt, FAD-Collagen I-Cu Salt, FAD-Collagen I-Pd Salt, FAD-Collagen I-Gd Salt, FAD-Collagen I-Er Salt, FAD-Collagen I-Mn Salt, FAD-Collagen I-Ag Salt, FAD-Collagen I-Co Salt, FAD-Collagen I-Zn Salt, FAD-Collagen I-Fe Salt, FAD-Collagen I-Ti Salt are also part of the invention and manufacturing.

The FAD-Collagen I-Au Salt and even more the FAD-Collagen I-HAuCl4*6H2O Salt is preferred.

### Collagen IV

According to the invention, Collagen IV-Salt of Au-FAD, Collagen IV-Salt of Cu-FAD, Collagen IV-Salt of Pd-FAD, Collagen IV-Salt of Gd-FAD, Collagen IV-Salt of Er-FAD, Collagen IV-Salt of Mn-FAD, Collagen IV-Salt of Ag-FAD, Collagen IV-Salt of Co-FAD, Collagen IV-Salt of Zn-FAD, Collagen IV-Salt of Fe-FAD, Collagen IV-Salt of Ti-FAD, is manufactured.

Collagen IV-Au-FAD Salt, and even more Collagen IV-Salt of HAuCl4*6H2O-FAD is preferred.

Compounds corresponding to Collagen IV-FAD-Au Salt, Collagen IV-FAD-Cu Salt, Collagen IV-FAD-Pd Salt, Collagen IV-FAD-Gd Salt, Collagen IV-FAD-Er Salt, Collagen IV-FAD-Mn Salt, Collagen IV-FAD Ag Salt, Collagen IV-FAD Co Salt, Collagen IV-FAD Zn Salt, Collagen IV-FAD Fe Salt, Collagen IV-FAD Ti Salt are also part of the invention and are manufactured. Collagen IV-FAD-Au Salt and even more Collagen IV-FAD-HAuCl4*6H2O Salt is preferred.

According to the invention an Au Salt-Collagen IV-FAD, Cu Salt-Collagen IV-FAD, Pd Salt-Collagen IV-FAD, Gd Salt-Collagen IV-FAD, ER Salt-Collagen IV-FAD, Mn Salt-Collagen IV-FAD, Ag Salt-Collagen IV-FAD, Co Salt-Collagen IV-FAD, Zn Salt-Collagen IV-FAD, Fe Salt-Collagen IV-FAD, Ti Salt-Collagen IV-FAD is manufactured. Au-Collagen IV-FAD Salt and even more HAuCl4*6H2O-collagen IV-FAD Salt is preferred.

Compounds corresponding to FAD-Collagen IV-Au Salt, FAD-Collagen IV-Cu Salt, FAD-Collagen IV-Pd Salt, FAD-Collagen IV-Gd Salt, FAD-Collagen IV-Er Salt, FAD-Collagen IV-Mn Salt, FAD-Collagen IV-Ag Salt, FAD-Collagen IV-Co Salt, FAD-Collagen IV-Zn Salt, FAD-Collagen IV-Fe Salt, FAD-Collagen IV-Ti Salt are also part of the invention. FAD-Collagen IV-Au Salt and even more FAD-Collagen IV-HAuCl4*6H2O Salt is preferred.

### Poly-Lacticide

POLY-LACTICIDE-Salt of Au-FAD, POLY-LACTICIDE-Salt of Cu-FAD, POLY-LACTICIDE-Salt of Pd-FAD, POLY-LACTICIDE-Salt of Gd-FAD, POLY-LACTICIDE-Salt of ER-FAD, POLY-LACTICIDE-Salt of Mn-FAD, POLY-LACTICIDE-Salt of Ag-FAD, POLY-LACTICIDE-Salt of Co-FAD, POLY-LACTICIDE-Salt of Zn-FAD, POLY-LACTICIDE-Salt of Fe-FAD, POLY-LACTICIDE-Salt of Ti-FAD, is manufactured. Au-FAD POLY-LACTICIDE Salt and even more so HAuCl4*6H2O-FAD POLY-LACTICIDE Salt is preferred.

Compounds corresponding to POLY-LACTICIDE-FAD-Au of Salt, POLY-LACTICIDE-FAD-Cu of Salt, POLY-LACTICIDE-FAD-Salt of Pd, POLY-LACTICIDE-FAD-Salt of Gd, POLY-LACTICIDE-FAD-Salt of Er, POLY-LACTICIDE-FAD-Salt of Mn, POLY-LACTICIDE-FAD Ag Salt, POLY-LACTICIDE-FAD Co Salt, POLY-LACTICIDE-FAD Zn Salt, POLY-LACTICIDE-FAD Fe Salt, POLY-LACTICIDE-FAD Ti Salt is also part of the invention and manufacturing. POLY-LACTICIDE-FAD-Salt of Au and even more POLY-LACTICIDE-FAD-Salt of HAuCl4*6H2O-is preferred.

Au Salt-POLY-LACTICIDE-FAD, Cu Salt-POLY-LACTICIDE-FAD, Pd Salt-POLY-LACTICIDE-FAD, Gd Salt-POLY-LACTICIDE-FAD, ER Salt-POLY-LACTICIDE-FAD, Mn Salt-POLY-LACTICIDE-FAD, Ag Salt-POLY-LACTICIDE-FAD, Co Salt-POLY-LACTICIDE-FAD, Zn Salt-POLY-LACTICIDE-FAD, Fe Salt-POLY-LACTICIDE-FAD, Ti Salt-POLY-LACTICIDE-FAD, is manufactured. Au-POLY-LACTICIDE-FAD Salt and even more HAuCl4*6H2O-POLY-LACTICIDE-FAD Salt is preferred.

Compounds corresponding to FAD-POLY-LACTICIDE-Au Salt, FAD-POLY-LACTICIDE-Cu Salt, FAD-POLY-LACTICIDE-Pd Salt, FAD-POLY-LACTICIDE-Gd Salt, FAD-POLY-LACTICIDE-Er Salt, FAD-POLY-LACTICIDE-Mn Salt, FAD-POLY-LACTICIDE-Ag Salt, FAD-POLY-LACTICIDE-Co Salt, FAD-POLY-LACTICIDE-Zn Salt, FAD-POLY-LACTICIDE-Fe Salt, FAD-POLY-LACTICIDE-Ti Salt are also part of the invention and manufacturing. FAD-POLY-LACTICIDE-Au Salt and even more FAD-POLY-LACTICIDE-HAuCl4*6H2O Salt is preferred.

BIS PHOPHONATE

BIS-PHOSPHONATE-Au Salt-FAD, BIS-PHOSPHONATE-Cu Salt-FAD, BIS-PHOSPHONATE-Pd Salt-FAD, BIS-PHOSPHONATE-Gd Salt-FAD, BIS-PHOSPHONATE-ER Salt-FAD, BIS-PHOSPHONATE Mn Salt-FAD, BIS-PHOSPHONATE-Ag Salt-FAD, BIS-PHOSPHONATE-Co Salt-FAD, BIS-PHOSPHONATE-Zn Salt-FAD, BIS-PHOSPHONATE-Fe Salt-FAD, BIS-PHOSPHONATE-Ti Salt-FAD, is manufactured. Au Salt BIS-PHOSPHONATE-FAD and even more HAuCl4*6H2O-BIS-PHOSPHONATE-FAD Salt is preferred.

Compounds corresponding to BIS-PHOSPHONATE-FAD-Au Salt, BIS-PHOSPHONATE-FAD-Cu Salt, BIS-PHOSPHONATE-FAD-Pd Salt, BIS-PHOSPHONATE-FAD-Gd Salt, BIS-PHOSPHONATE-FAD-Er Salt, BIS-PHOSPHONATE-FAD-Mn Salt, BIS-PHOSPHONATE-FAD Ag Salt, BIS-PHOSPHONATE-FAD Co Salt, BIS-PHOSPHONATE-FAD Zn Salt, BIS-PHOSPHONATE-FAD Fe Salt, BIS-PHOSPHONATE-FAD Ti Salt is also part of the invention and manufacturing. BIS-PHOSPHONATE-FAD-Salt of Au and even more BIS-PHOSPHONATE-FAD-Salt of HAuCl4*6H2O is preferred.

An Au Salt-BIS-PHOSPHONATE-FAD, Cu Salt-BIS-PHOSPHONATE-FAD, Pd Salt-BIS-PHOSPHONATE-FAD, Gd Salt-BIS-PHOSPHONATE-FAD, ER Salt-BIS-PHOSPHONATE-FAD, Mn Salt-BIS-PHOSPHONATE-FAD, Ag Salt-BIS-PHOSPHONATE-FAD, Co Salt-BIS-PHOSPHONATE-FAD, Zn Salt-BIS-PHOSPHONATE-FAD, Fe Salt-BIS-PHOSPHONATE-FAD, Ti Salt-BIS-PHOSPHONATE-FAD, is manufactured. Au-BIS-PHOSPHONATE-FAD Salt and even more HAuCl4*6H2O-BIS-PHOSPHONATE-FAD Salt is preferred.

Compounds corresponding to FAD-BIS-PHOSPHONATE-Au Salt, FAD-BIS-PHOSPHONATE-Cu Salt, FAD-BIS-PHOSPHONATE-Pd Salt, FAD-BIS-PHOSPHONATE-Gd Salt, FAD-BIS-PHOSPHONATE-Er Salt, FAD-BIS-PHOSPHONATE-Mn Salt, FAD-BIS-PHOSPHONATE-Ag Salt, FAD-BIS-PHOSPHONATE-Co Salt, FAD-BIS-PHOSPHONATE-Zn Salt, FAD-BIS-PHOSPHONATE-Fe Salt, FAD-BIS-PHOSPHONATE-Ti Salt are also part of the invention and manufacturing. FAD-BIS-PHOSPHONATE-Salt of Au and even more FAD-BIS-PHOSPHONATE-Salt of HAuCl4*6H2O-FAD is preferred.

### Gelatine

According to the invention an object GELATINE-Au Salt-FAD, GELATINE-Cu Salt-FAD, GELATINE-Pd Salt-FAD, GELATINE-Gd Salt-FAD, GELATINE-ER Salt-FAD, GELATINE Mn Salt-FAD, GELATINE Ag Salt-FAD, GELATINE Co Salt-FAD, GELATINE Zn Salt-FAD, GELATINE Fe Salt-FAD, GELATINE Ti-FAD Salt is manufactured. GELATINE-AU-FAD Salt and even more GELATINE-HAuCl4*6H2O-FAD Salt is preferred.

According to the invention, GELATINE-FAD-Salt of Au, GELATINE-FAD-Salt of Cu, GELATINE-FAD-Salt of Pd, GELATINE-FAD-Salt of Gd, GELATINE-FAD-Salt of Er, GELATINE-FAD-Salt of Mn, GELATINE-FAD-Salt of Ag, GELATINE-FAD-Salt of Co, GELATINE-FAD-Salt of Zn, GELATINE-FAD-Salt of Fe, GELATINE-FAD Ti Salt is also part of the invention and is manufactured. GELATINE-FAD-Salt of Au and even more GELATINE-FAD-Salt of HAuCl4*6H2O is preferred.

One Au Salt-GELATINE-FAD, Cu Salt-GELATINE-FAD, Pd Salt-GELATINE-FAD, Gd Salt-GELATINE-FAD, ER Salt-GELATINE-FAD, Mn Salt-GELATINE-FAD, Ag Salt-GELATINE-FAD, Co Salt-GELATINE-FAD, Zn Salt-GELATINE-FAD, Fe Salt-GELATINE-FAD, Ti Salt-GELATINE-FAD, is manufactured. Au-GELATINE-FAD Salt and even more GELATINE-SALT from HAuCl4*6H2O-FAD is preferred.

A compound corresponding to FAD-GELATINE-Au Salt, FAD-GELATINE-Cu Salt, FAD-GELATINE-Pd Salt, FAD-GELATINE-Gd Salt, FAD-GELATINE-Er Salt, FAD-GELATINE-Mn Salt, FAD-GELATINE-Ag Salt, FAD-GELATINE-Co Salt, FAD-GELATINE-Zn Salt, FAD-GELATINE-Fe Salt, FAD-GELATINE-Ti Salt is also part of the invention and is also manufactured. FAD-GELATINE-Salt of Au and even more FAD-GELATINE- the Salt of HAuCl4*6H2O is preferred.

### Maltodextrin

A compound corresponding to MALTODEXTRIN-Au Salt-FAD, MALTODEXTRIN-Cu Salt-FAD, MALTODEXTRIN-Pd Salt-FAD, MALTODEXTRIN-Gd Salt-FAD, MALTODEXTRIN-Er Salt-FAD, MALTODEXTRIN-Mn Salt-FAD, MALTODEXTRIN-Ag Salt-FAD, MALTODEXTRIN Co Salt-FAD, MALTODEXTRIN Zn Salt FAD, MALTODEXTRIN Fe Salt-FAD, MALTODEXTRIN Ti Salt-FAD, is part of the invention and is manufactured in particle, micro or nanoparticle form. MALTODEXTRIN-Salt from Au-FAD and even more so MALTODEXTRIN-Salt from HAuCl4*6H2O-FAD is preferred.

Compounds corresponding to MALTODEXTRIN-FAD-Salt of Au, MALTODEXTRIN-FAD-Salt of Cu, MALTODEXTRIN-FAD-Salt of Pd, MALTODEXTRIN-FAD-Salt of Gd, MALTODEXTRIN-FAD-Salt of Er, MALTODEXTRIN-FAD-Salt of Mn, MALTODEXTRIN-FAD-Salt of Ag, MALTODEXTRIN-FAD-Salt of Co, MALTODEXTRIN-FAD-Salt of Zn, MALTODEXTRIN-FAD-Salt of Fe, MALTODEXTRIN-FAD Salt of Ti are also part of the invention and manufacturing. MALTODEXTRIN-FAD-Salt of Au- and even more MALTODEXTRIN-FAD-Salt of HAuCl4*6H2O-is preferred.

According to the invention, an Au Salt-MALTODEXTRIN-FAD, Cu Salt-MALTODEXTRIN-FAD, Pd Salt-MALTODEXTRIN-FAD, Gd Salt-MALTODEXTRIN-FAD, ER Salt-MALTODEXTRIN-FAD, Mn Salt-MALTODEXTRIN-FAD, Ag Salt-MALTODEXTRIN-FAD, Co Salt-MALTODEXTRIN-FAD, Zn Salt-MALTODEXTRIN-FAD, Fe Salt-MALTODEXTRIN-FAD, Ti Salt-MALTODEXTRIN-FAD, is manufactured. Au-MALTODEXTRIN-FAD Salt and even more HAuCl4*6H2O-MALTODEXTRIN-FAD Salt is preferred.

Compounds corresponding to FAD-MALTODEXTRIN-Au Salt, FAD-MALTODEXTRIN-Cu Salt, FAD-MALTODEXTRIN-Pd Salt, FAD-MALTODEXTRIN-Gd Salt, FAD-MALTODEXTRIN-Er Salt, FAD-MALTODEXTRIN-Mn Salt, FAD-MALTODEXTRIN-Ag Salt, FAD-MALTODEXTRIN-Co Salt, FAD-MALTODEXTRIN-Zn Salt, FAD-MALTODEXTRIN-Fe Salt, FAD-MALTODEXTRIN-Ti Salt are also part of the invention and manufacturing. FAD-MALTODEXTRIN-Au Salt and even more FAD-MALTODEXTRIN-HAuCl4*6H2O Salt is preferred.

### Poly-L-Lysine

According to the invention a POLY-L-LYSINE-Salt of Au-FAD, POLY-L-LYSINE-Salt of Cu-FAD, POLY-L-LYSINE-Salt of Pd-FAD, POLY-L-LYSINE-Salt of Gd-FAD, POLY-L-LYSINE-Salt of ER-FAD, POLY-L-LYSINE- the Salt of Mn-FAD, POLY-L-LYSINE- the Salt of Ag-FAD, POLY-L-LYSINE- the Salt of Co-FAD, POLY-L-LYSINE- the Salt of Zn-FAD, POLY-L-LYSINE- the Salt of Fe-FAD, POLY-L-LYSINE- the Salt of Ti-FAD, is manufactured. POLY-L-LYSINE- the Salt of Au-FAD and even more POLY-L-LYSINE- the Salt of HAuCl4*6H2O-FAD is preferred. A compound corresponding to POLY-L-LYSINE-FAD-Salt of Au, POLY-L-LYSINE-FAD-Salt of Cu, POLY-L-LYSINE-FAD-Salt of Pd, POLY-L-LYSINE-FAD-Salt of Gd, POLY-L-LYSINE-FAD-Salt of Er, POLY-L-LYSINE-FAD-Salt of Mn, POLY-L-LYSINE-FAD Salt of Ag, POLY-L-LYSINE-FAD Salt of Co, POLY-L-LYSINE-FAD Salt of Zn, POLY-L-LYSINE-FAD Salt of Fe, POLY-L-LYSINE-FAD Ti Salt is also part of the invention and is manufactured. POLY-L-LYSINE-FAD-Salt of Au and even more POLY-L-LYSINE-FAD-Salt of HAuCl4*6H2O is preferred.

According to the invention an Au Salt-POLY-L-LYSINE-FAD, Cu Salt-POLY-L-LYSINE-FAD, Pd Salt-POLY-L-LYSINE-FAD, Gd Salt-POLY-L-LYSINE-FAD, ER Salt-POLY-L-LYSINE-FAD, Mn Salt-POLY-L-LYSINE-FAD, Ag Salt-POLY-L-LYSINE-FAD, Co Salt-POLY-L-LYSINE-FAD, Zn Salt-POLY-L-LYSINE-FAD, Fe Salt-POLY-L-LYSINE-FAD, Ti Salt-POLY-L-LYSINE-FAD, is manufactured. Au-POLY-L-LYSINE-FAD Salt and even more HAuCl4*6H2O-POLY-L-LYSINE-FAD Salt is preferred.

According to the invention a FAD-POLY-L-LYSINE-Au Salt, FAD-POLY-L-LYSINE-Cu Salt, FAD-POLY-L-LYSINE-Pd Salt, FAD-POLY-L-LYSINE-Gd Salt, FAD-POLY-L-LYSINE-Er Salt, FAD-POLY-L-LYSINE-Mn Salt, FAD-POLY-L-LYSINE-Ag Salt, FAD-POLY-L-LYSINE-Co Salt, FAD-POLY-L-LYSINE-Zn Salt, FAD-POLY-L-LYSINE-Fe Salt, FAD-POLY-L-LYSINE-Ti Salt is also part of the invention and is manufactured. FAD-POLY-L-LYSINE-Salt of Au and even more FAD-POLY-L-LYSINE-Salt of HAuCl4*6H2O is preferred.

### Poly-L-ornithine

According to the invention a POLY-L-ORNITHINE-Salt of Au-FAD, POLY-L-ORNITHINE-Salt of Cu-FAD, POLY-L-ORNITHINE-Salt of Pd-FAD, POLY-L-ORNITHINE-Salt of Gd-FAD, POLY-L-ORNITHINE-Salt of ER-FAD, POLY-L-ORNITHINE-Salt of Mn-FAD, POLY-L-ORNITHINE-Salt of Ag-FAD, POLY-L-ORNITHINE-Salt of Co-FAD, POLY-L-ORNITHINE-Salt of Zn-FAD, POLY-L-ORNITHINE-Salt of Fe-FAD, POLY-L-ORNITHINE-Ti-FAD Salt, is manufactured. Poly-L-ornithine Salt of Au-FAD and even more Poly-L-ornithine Salt of HAuCl4*6H2O-FAD is preferred.

A compound corresponding to POLY-L-ORNITHINE-FAD-Salt of Au, POLY-L-ORNITHINE-FAD-Salt of Cu, POLY-L-ORNITHINE-FAD-Salt of Pd, POLY-L-ORNITHINE-FAD-Salt of Gd, POLY-L-ORNITHINE-FAD-Salt of Er, POLY-L-ORNITHINE-FAD-Salt of Mn, POLY-L-ORNITHINE-FAD-Salt of Ag, POLY-L-ORNITHINE-FAD-Salt of Co, POLY-L-ORNITHINE-FAD-Salt of Zn, POLY-L-ORNITHINE-FAD-Salt of Fe, POLY-L-ORNITHINE-FAD Ti Salt is also part of the invention and is manufactured. Poly-L-ornithine-FAD-Salt from Au and even more Poly-L-ornithine-FAD-Salt from HAuCl4*6H2O-is preferred.

According to the invention an Au-POLY-L-ORNITHINE-FAD Salt, Cu-POLY-L-ORNITHINE-FAD Salt, Pd-POLY-L-ORNITHINE-FAD Salt, Gd-POLY-L-ORNITHINE-FAD Salt, ER-POLY-L-ORNITHINE-FAD Salt, Mn-POLY-L-ORNITHINE-FAD Salt, Ag-POLY-L-ORNITHINE-FAD Salt, Co-POLY-L-ORNITHINE-FAD Salt, Zn-POLY-L-ORNITHINE-FAD Salt, Fe-POLY-L-ORNITHINE-FAD Salt, Ti-POLY-L-ORNITHINE-FAD Salt, is manufactured. Au-Poly-L-ornithine-FAD Salt and even more HAuCl4*6H2O-Poly-L-ornithine-FAD Salt is preferred.

According to the invention a FAD-POLY-L-ORNITHINE-Au Salt, FAD-POLY-L-ORNITHINE-Cu Salt, FAD-POLY-L-ORNITHINE-Pd Salt, FAD-POLY-L-ORNITHINE-Gd Salt, FAD-POLY-L-ORNITHINE-Er Salt, FAD-POLY-L-ORNITHINE-Mn Salt, FAD-POLY-L-ORNITHINE-Ag Salt, FAD-POLY-L-ORNITHINE-Co Salt, FAD-POLY-L-ORNITHINE-Zn Salt, FAD-POLY-L-ORNITHINE-Fe Salt, FAD-POLY-L-ORNITHINE-Ti Salt is also part of the invention and is manufactured. FAD-POLY-L-ORNITHINE- the Salt of Au and even more FAD-POLY-L-ORNITHINE- the Salt of HAuCl4*6H2O is preferred.

According to the invention a POLY-L-arginine- the Salt of Au-FAD, POLY-L-arginine-Salt of Cu-FAD, POLY-L-arginine-Salt of Pd-FAD, POLY-L-arginine- the Salt of Gd-FAD, POLY-L-arginine-Salt of ER-FAD, POLY-L-arginine- the Salt of Mn-FAD, POLY-L-arginine- the Salt of Ag-FAD, POLY-L-arginine- the Salt of Co-FAD, POLY-L-arginine- the Salt of Zn-FAD, POLY-L-arginine- the Salt of Fe-FAD, POLY-L-arginine- the Salt of Ti-FAD, is manufactured. POLY-L-arginine- the Salt of Au-FAD and even more POLY-L-arginine- the Salt of HAuCl4*6H2O-FAD is preferred.

According to the invention a POLY-L-arginine-FAD-Salt of Au, POLY-L-arginine-FAD-Salt of Cu, POLY-L-arginine-FAD-Salt of Pd, POLY-L-arginine-FAD-Salt of Gd, POLY-L-arginine-FAD-Salt of Er, POLY-L-arginine-FAD-Salt of Mn, POLY-L-arginine-FAD Salt of Ag, POLY-L-arginine-FAD Salt of Co, POLY-L-arginine-FAD Salt of Zn, POLY-L-arginine-FAD Salt of Fe, POLY-L-arginine-FAD Ti Salt is also part of the invention and is manufactured. POLY-L-arginine-FAD-Salt of Au and even more POLY-L-arginine-FAD-Salt of HAuCl4*6H2O is preferred.

According to the invention a Au Salt POLY-L-arginine-FAD, Cu Salt POLY-L-arginine-FAD, Pd Salt POLY-L-arginine-FAD, Gd Salt POLY-L-arginine-FAD, Er Salt POLY-L-arginine-FAD, Mn Salt POLY-L-arginine-FAD, Ag Salt POLY-L-arginine-FAD, Co Salt POLY-L-arginine-FAD, Zn Salt POLY-L-arginine-FAD, Fe Salt POLY-L-arginine-FAD, Ti Salt POLY-L-arginine-FAD, is manufactured. The Au-POLY-L-arginine-FAD Salt and even more the HAuCl4*6H2O-POLY-L-arginine-FAD Salt is preferred.

According to the invention a FAD-POLY-L-arginine-Au Salt, FAD-POLY-L-arginine-Cu Salt, FAD-POLY-L-arginine-Pd Salt, FAD-POLY-L-arginine-Gd Salt, FAD-POLY-L-arginine-Er Salt, FAD-POLY-L-arginine-Mn Salt, FAD-POLY-L-arginine-Ag Salt, FAD-POLY-L-arginine-Co Salt, FAD-POLY-L-arginine-Zn Salt, FAD-POLY-L-arginine-Fe Salt, FAD-POLY-L-arginine-Ti Salt is also part of the invention and is manufactured. The FAD-POLY-L-arginine- the Salt of Au and even more the FAD-POLY-L-arginine- the Salt of HAuCl4*6H2O is preferred.

According to the invention a LAURYL-POLYGLUCOSE-Au-FAD Salt, LAURYL-POLYGLUCOSE-Cu-FAD Salt, LAURYL-POLYGLUCOSE-Pd-FAD Salt, LAURYL-POLYGLUCOSE-Gd-FAD Salt, LAURYL-POLYGLUCOSE-ER-FAD Salt, LAURYL-POLYGLUCOSE-Mn-FAD Salt, LAURYL-POLYGLUCOSE-Ag-FAD Salt, LAURYL-POLYGLUCOSE-Co-FAD Salt, LAURYL-POLYGLUCOSE-Zn-FAD Salt, LAURYL-POLYGLUCOSE-Fe-FAD Salt, LAURYL-POLYGLUCOSE-Ti-FAD Salt, is manufactured. LAURYL-POLYGLUCOSE-Salt of Au-FAD and even more LAURYL-POLYGLUCOSE-Salt of HAuCl4*6H2O-FAD is preferred.

According to the invention a LAURYL-POLYGLUCOSE-FAD-Salt of Au, LAURYL-POLYGLUCOSE-FAD-Salt of Cu, LAURYL-POLYGLUCOSE-FAD-Salt of Pd, LAURYL-POLYGLUCOSE-FAD-Salt of Gd, LAURYL-POLYGLUCOSE-FAD-Salt of Er, LAURYL-POLYGLUCOSE-FAD-Salt of Mn, LAURYL-POLYGLUCOSE-FAD Ag Salt, LAURYL-POLYGLUCOSE-FAD-Salt of Co, LAURYL-POLYGLUCOSE-FAD-Salt of Zn, LAURYL-POLYGLUCOSE-FAD-Salt of Fe, LAURYL-POLYGLUCOSE-FAD Ti Salt is also part of the invention and is manufactured. LAURYL-POLYGLUCOSE-FAD-Salt of Au and even more LAURYL-POLYGLUCOSE-FAD-Salt of HAuCl4*6H2O are preferred.

According to the invention an Au Salt-LAURYL-POLYGLUCOSE-FAD, Cu Salt-LAURYL-POLYGLUCOSE-FAD, Pd Salt-LAURYL-POLYGLUCOSE-FAD, Gd Salt-LAURYL-POLYGLUCOSE-FAD, ER Salt-LAURYL-POLYGLUCOSE-FAD, Mn Salt-LAURYL-POLYGLUCOSE-FAD, Ag Salt-LAURYL-POLYGLUCOSE-FAD, Co Salt-LAURYL-POLYGLUCOSE-FAD, Zn Salt-LAURYL-POLYGLUCOSE-FAD, Fe Salt-LAURYL-POLYGLUCOSE-FAD, Ti-LAURYL-POLYGLUCOSE-FAD Salt, is manufactured. The Au-LAURYL-POLYGLUCOSE-FAD Salt and even more the HAuCl4*6H2O-LAURYL-POLYGLUCOSE-FAD Salt is preferred.

According to the invention a FAD-LAURYL-POLYGLUCOSE-Au Salt, FAD-LAURYL-POLYGLUCOSE-Cu Salt, FAD-LAURYL-POLYGLUCOSE-Pd Salt, FAD-LAURYL-POLYGLUCOSE-Gd Salt, FAD-LAURYL-POLYGLUCOSE-Er Salt, FAD-LAURYL-POLYGLUCOSE-Mn Salt, FAD-LAURYL-POLYGLUCOSE-Ag Salt, FAD-LAURYL-POLYGLUCOSE-Co Salt, FAD-LAURYL-POLYGLUCOSE-Zn Salt, FAD-LAURYL-POLYGLUCOSE-Fe Salt, FAD-LAURYL-POLYGLUCOSE-Ti Salt is also part of the invention and is manufactured. FAD-LAURYL-POLYGLUCOSE-AuCI Salt and even more FAD-LAURYL-POLYGLUCOSE-HAuCl4*6H2O-is preferred.

According to the invention, a hyaluronic acid-Salt of Au-FAD, hyaluronic acid-Salt of Cu-FAD, hyaluronic acid-Salt of Pd-FAD, hyaluronic acid-Salt of Gd-FAD, hyaluronic acid-Salt of ER-FAD, hyaluronic acid-Salt of Mn-FAD, hyaluronic acid-Salt of Ag-FAD, hyaluronic acid-Salt of Co-FAD, hyaluronic acid-Salt of Zn-FAD, hyaluronic acid-Salt of Fe-FAD, hyaluronic acid-Salt of Ti-FAD, is manufactured. The hyaluronic acid-Salt of Au-FAD, and even more the hyaluronic acid-Salt of HAuCl4*6H2O-FAD is preferred.

Corresponding compounds are hyaluronic acid-FAD-Salt Au, hyaluronic acid-FAD-Salt Cu, hyaluronic acid-FAD-Salt Pd, hyaluronic acid-FAD-Salt Gd, hyaluronic acid-FAD-Salt Er, hyaluronic acid-FAD-Salt Mn, Hyaluronic acid-FAD Salt of Ag, hyaluronic acid-FAD Salt of Co, hyaluronic acid-FAD Salt of Zn, hyaluronic acid-FAD Salt of Fe, hyaluronic acid-FAD Salt of Ti is also part of the invention. Hyaluronic acid-FAD-Salt of Au, and even more hyaluronic acid-FAD-Salt of HAuCl4*6H2O are preferred.

According to the invention, a Salt of Au-hyaluronic acid-FAD, Salt of Cu-hyaluronic acid-FAD, Salt of Pd-hyaluronic acid-FAD, Salt of Gd-hyaluronic acid-FAD, Salt of ER-hyaluronic acid-FAD, Mn-Salt of hyaluronic acid-FAD, Ag-Salt of hyaluronic acid-FAD, Co-Salt of hyaluronic acid-FAD, Zn-Salt of hyaluronic acid-FAD, Fe-Salt of hyaluronic acid-FAD, Ti-Salt of hyaluronic acid-FAD, is manufactured. Au-FAD Salt of hyaluronic acid and even more HAuCl4*6H2O-FAD Salt of hyaluronic acid is preferred.

Compounds corresponding to FAD-hyaluronic acid-Au Salt, FAD-hyaluronic acid-Cu Salt, FAD-hyaluronic acid-Pd Salt, FAD-hyaluronic acid-Gd Salt, FAD-hyaluronic acid-Er Salt, FAD-hyaluronic acid-Mn Salt, FAD-hyaluronic acid-Ag Salt, FAD-hyaluronic acid-Co Salt, FAD-hyaluronic acid-Zn Salt, FAD-hyaluronic acid-Fe Salt, FAD-hyaluronic acid-Ti Salt are also part of the invention and manufacturing. FAD-hyaluronic acid-Au Salt, and even more FAD-hyaluronic acid-HAuCl4*6H2O-FAD is preferred.

### Cellulose

According to the invention, a combination of CELLULOSE-Salt of Au-FAD, CELLULOSE-Salt of Cu-FAD, CELLULOSE-Salt of Pd-FAD, CELLULOSE-Salt of Gd-FAD, CELLULOSE-Salt of ER-FAD, CELLULOSE-Salt of Mn-FAD, CELLULOSE-Salt of Ag-FAD, CELLULOSE-Salt of Co-FAD, CELLULOSE-Salt of Zn-FAD, CELLULOSE-Salt of Fe-FAD, CELLULOSE-Salt of Ti-FAD, is manufactured. The CELLULOSE-Salt of Au-FAD, and even more the CELLULOSE-Salt of HAuCl4*6H2O-FAD is preferred.

Compounds corresponding to CELLULOSE-FAD-Salt of Au, CELLULOSE-FAD-Salt of Cu, CELLULOSE-FAD-Salt of Pd, CELLULOSE-FAD-Salt of Gd, CELLULOSE-FAD-Salt of Er, CELLULOSE-FAD-Salt of Mn, CELLULOSE-FAD Salt of Ag, CELLULOSE-FAD Salt of Co, CELLULOSE-FAD Salt of Zn, CELLULOSE-FAD Salt of Fe, CELLULOSE-FAD Ti Salt are also part of the invention. CELLULOSE-FAD-Salt of Au and even more CELLULOSE-FAD-Salt of HAuCl4*6H2O are preferred.

According to the invention, Au Salt-CELLULOSE-FAD, Cu Salt-CELLULOSE-FAD, Pd Salt-CELLULOSE-FAD, Gd Salt-CELLULOSE-FAD, ER Salt-CELLULOSE-FAD, Mn Salt-CELLULOSE-FAD, Ag Salt-CELLULOSE-FAD, Co Salt-CELLULOSE-FAD, Zn Salt-CELLULOSE-FAD, Fe Salt-CELLULOSE-FAD, Ti Salt-CELLULOSE-FAD, is manufactured. Au-FAD-CELLULOSE Salt and even more HAuCl4*6H2O-FAD-CELLULOSE Salt is preferred.

Compounds corresponding to FAD-CELLULOSE-Au Salt, FAD-CELLULOSE-Cu Salt, FAD-CELLULOSE-Pd Salt, FAD-CELLULOSE-Gd Salt, FAD-CELLULOSE-Er Salt, FAD-CELLULOSE-Mn Salt, FAD-CELLULOSE-Ag Salt, FAD-CELLULOSE-Co Salt, FAD-CELLULOSE-Zn Salt, FAD-CELLULOSE-Fe Salt, FAD-CELLULOSE-Ti Salt are also part of the invention and manufacturing. The FAD-CELLULOSE-Salt of Au-, and even more the FAD-CELLULOSE-Salt of HAuCl4*6H2O-FAD is preferred.

### Elastine

According to the invention, an ELASTINE-Salt of Au-FAD, ELASTINE-Salt of Cu-FAD, ELASTINE-Salt of Pd-FAD, ELASTINE-Salt of Gd-FAD, ELASTINE-Salt of ER-FAD, ELASTINE-Salt of Mn-FAD, ELASTINE-Salt of Ag-FAD, ELASTINE-Salt of Co-FAD, ELASTINE-Salt of Zn-FAD, ELASTINE-Salt of Fe-FAD, ELASTINE-Salt of Ti-FAD, is manufactured. ELASTINE-Salt of Au-FAD, and even more ELASTINE-Salt of HAuCl4*6H2O-FAD is preferred.

Compounds corresponding to ELASTINE-FAD-Salt of Au, ELASTINE-FAD-Salt of Cu, ELASTINE-FAD-Salt of Pd, ELASTINE-FAD-Salt of Gd, ELASTINE-FAD-Salt of Er, ELASTINE-FAD-Salt of Mn, ELASTINE-FAD-Salt of Ag, ELASTINE-FAD-Salt of Co, ELASTINE-FAD Salt of Zn, ELASTINE-FAD Salt of Fe, ELASTINE-FAD Ti Salt are also part of the invention. The ELASTINE-FAD Salt of Au, and even more the ELASTINE-FAD Salt of HAuCl4*6H2O is preferred.

According to the invention, Au Salt-ELASTINE-FAD, Cu Salt-ELASTINE-FAD, Pd Salt-ELASTINE-FAD, Gd Salt-ELASTINE-FAD, ER Salt-ELASTINE-FAD, Mn Salt-ELASTINE-FAD, Ag Salt-ELASTINE-FAD, Co Salt-ELASTINE-FAD, Zn Salt-ELASTINE-FAD, Fe Salt-ELASTINE-FAD, Ti Salt-ELASTINE-FAD, is manufactured. Au-FAD-ELASTINE Salt and even more HAuCl4*6H2O-FAD-ELASTINE Salt is preferred.

Compounds corresponding to FAD-ELASTINE-Au Salt, FAD-ELASTINE-Cu Salt, FAD-ELASTINE-Pd Salt, FAD-ELASTINE-Gd Salt, FAD-ELASTINE-Er Salt, FAD-ELASTINE-Mn Salt, FAD-ELASTINE-Ag Salt, FAD-ELASTINE-Co Salt, FAD-ELASTINE-Zn Salt, FAD-ELASTINE-Fe Salt, FAD-ELASTINE-Ti Salt are also part of the invention and manufacturing. The FAD-ELASTINE-Salt of Au-, and even more the FAD-ELASTINE-Salt of HAuCl4*6H2O-FAD is preferred.

Corresponding to an embodiment, glycogen-Salt of Au-FAD, glycogen-Salt of Cu-FAD, glycogen-Salt of Pd-FAD, glycogen-Salt of Gd-FAD, glycogen-Salt of ER-FAD, glycogen-Salt of Mn-FAD, glycogen-Salt of Ag-FAD, glycogen-Salt of Co-FAD, glycogen-Salt of Zn-FAD, glycogen-Salt of Fe-FAD, glycogen-Salt of Ti-FAD, is manufactured. Au-FAD glycogen-Salt and even more HAuCl4*6H2O-FAD glycogen-Salt are preferred.

Corresponding compounds of glycogen-FAD-Au Salt, glycogen-FAD-Cu Salt, glycogen-FAD-Pd Salt, glycogen-FAD-Gd Salt, glycogen-FAD-Er Salt, glycogen-FAD-Mn Salt, Glycogen-FAD-Ag Salt, glycogen-FAD-Co Salt, glycogen-FAD-Zn Salt, glycogen-FAD-Fe Salt, glycogen-FAD-Ti Salt are also part of the invention. Glycogen-FAD-Au Salt and even more glycogen-FAD-HAuCl4*6H2O-FAD Salt is preferred.

According to the invention, Au Salt-Glycogen-FAD, Cu Salt-Glycogen-FAD, Pd Salt-Glycogen-FAD, Gd Salt-Glycogen-FAD, ER Salt-Glycogen-FAD, Mn Salt-Glycogen-FAD, Ag Salt-Glycogen-FAD, Co Salt-Glycogen-FAD, Zn Salt-Glycogen-FAD, Fe Salt Glycogen-FAD, Ti Sal-Glycogen-FAD is manufactured. Au-Glycogen-FAD Salt, and even more HAuCl4*6H2O-Glycogen-FAD Salt is preferred.

Corresponding to an embodiment, starch-Au Salt-FAD, starch-Cu Salt-FAD, starch-Pd Salt-FAD, starch-Gd Salt-FAD, starch-ER Salt-FAD, starch-Mn Salt-FAD, starch-Ag Salt-FAD, starch-Co Salt-FAD, starch-Zn-Salt FAD, starch-Fe-Salt FAD, Ti-FAD starch-Salt, is manufactured. Starch-Salt of Au-FAD and even more starch-Salt of HAuCl4*6H2O-FAD are preferred.

Corresponding compounds of starch-FAD-Au Salt, starch-FAD-Cu Salt, starch-FAD-Pd Salt, starch-FAD-Gd Salt, starch-FAD-Er Salt, starch-FAD-Mn Salt, starch-FAD-Ag Salt, starch-FAD-Co Salt, starch-FAD-Zn Salt, starch-FAD-Fe Salt, starch-FAD-Ti Salt are also part of the invention. Starch-FAD-Salt of Au, and even more starch-FAD-Salt of HAuCl4*6H2O-FAD is preferred.

According to the invention, an Au Salt-starch-FAD, Cu Salt-starch-FAD, Pd-Salt-starch-FAD, Gd-Salt-starch-FAD, ER Salt-starch-FAD, Mn-Starch Salt-FAD, Ag-Starch Salt-FAD, Co-Starch Salt-FAD, Zn-Starch Salt-FAD, Fe-Starch Salt-FAD, Ti-Starch Salt-FAD, is manufactured. Au-Starch-FAD Salt, and even more HAuCl4*6H2O-Starch-FAD Salt is preferred.

Compounds corresponding to FAD-Starch-Au Salt, FAD-Starch-Cu Salt, FAD-Starch-Pd Salt, FAD-Starch-Gd Salt, FAD-Starch-Er Salt, FAD-Starch-Mn Salt, FAD-Starch-Ag Salt, FAD-Starch-Co Salt, FAD-Starch-Zn Salt, FAD-Starch-Fe Salt, FAD-Starch-Ti Salt are also part of the invention. The FAD-Au-Starch, and even more the FAD-HAuCl4*6H2O-Starch is preferred.

Corresponding to an embodiment, saccharose-Salt of Au-FAD, saccharose-Salt of Cu-FAD, saccharose-Salt of Pd-FAD, saccharose-Salt of Gd-FAD, saccharose-Salt of ER-FAD, saccharose-Salt of Mn-FAD, saccharose-Salt of Ag-FAD, saccharose-Salt of Co-FAD, saccharose-Salt of Zn-FAD, saccharose-Salt of Fe-FAD, saccharose-Salt of Ti-FAD, is manufactured. Au-FAD saccharose-Salt and even more HAuCl4*6H2O-FAD saccharose-Salt are preferred.

Compounds corresponding to saccharose-FAD-Salt of Au, saccharose-FAD-Salt of Cu, saccharose-FAD-Salt of Pd, saccharose-FAD-Salt of Gd, saccharose-FAD-Salt of Er, saccharose-FAD-Salt of Mn, saccharose-FAD-Salt of Ag, saccharose-FAD-Salt of Co, saccharose-FAD-Salt of Zn, saccharose-FAD-Salt of Fe, saccharose-FAD-Salt of Ti are also part of the invention. The saccharose-FAD-Salt of Au, and even more the saccharose-FAD-Salt of HAuCl4*6H2O-FAD is preferred.

According to the invention, Au Salt-saccharose-FAD, Cu Salt-saccharose-FAD, Pd Salt-saccharose-FAD, Gd Salt-saccharose-FAD, ER Salt-saccharose-FAD, Mn Salt-Saccharose-FAD, Ag Salt-Saccharose-FAD, Co Salt-Saccharose-FAD, Zn Salt-Saccharose-FAD, Fe Salt-Saccharose-FAD, Ti-Saccharose-FAD Salt is manufactured. Au-Saccharose-FAD Salt, and even more HAuCl4*6H2O-Saccharose-FAD Salt is preferred.

Compounds corresponding to FAD-Saccharose-Salt of Au, FAD-Saccharose-Salt of Cu, FAD-Saccharose-Salt of Pd, FAD-Saccharose-Salt of Gd, FAD-Saccharose-Salt of Er, FAD-Saccharose-Salt of Mn, FAD-Saccharose-Salt of Ag, FAD-Saccharose-Salt of Co, FAD-Saccharose-Salt of Zn, FAD-Saccharose-Salt of Fe, FAD-Saccharose-Salt of Ti are also part of the invention. FAD-Saccharose-Salt of Au and even more FAD-Saccharose-Salt of HAuCl4*6H2O is preferred.

Corresponding to an embodiment, lactose-Au Salt-FAD, lactose-Cu Salt-FAD, lactose-Pd Salt-FAD, lactose-Gd Salt-FAD, lactose-ER Salt-FAD, lactose-Mn Salt-FAD, lactose-Ag Salt-FAD, lactose-Co Salt-FAD, lactose-Zn Salt-FAD, lactose-Fe Salt-FAD, lactose-Ti-FAD Salt is produced. Au-FAD lactose-Salt and even more HAuCl4*6H2O-FAD lactose-Salt are preferred.

Compounds corresponding to lactose-FAD Salt Au, lactose-FAD Salt Cu, lactose-FAD Salt Pd, lactose-FAD Salt Gd, lactose-FAD Salt Er, lactose-FAD Salt Mn, lactose-FAD Salt Ag, lactose-FAD Salt Co, lactose-FAD Salt Zn, lactose-FAD Salt Fe, lactose-FAD Salt Ti are also part of the invention. Au lactose-FAD-Salt, and even more HAuCl4*6H2O-FAD lactose-FAD-Salt is preferred.

According to the invention, an Au Salt-Lactose-FAD, Cu Salt-Lactose-FAD, Pd Salt-Lactose-FAD, Gd Salt-Lactose-FAD, ER Salt-Lactose-FAD, Mn Salt-Lactose-FAD, Ag Salt-Lactose-FAD, Co Salt-Lactose-FAD, Zn Salt-Lactose-FAD, Fe Salt-Lactose-FAD, Ti-Lactose-FAD Salt is manufactured. Au-Lactose-FAD Salt and even more HAuCl4*6H2O-Lactose-FAD Salt is preferred.

Compounds corresponding to FAD-Lactose-Salt of Au, FAD-Lactose-Salt of Cu, FAD-Lactose-Salt of Pd, FAD-Lactose-Salt of Gd, FAD-Lactose-Salt of Er, FAD-Lactose-Salt of Mn, FAD-Lactose-Salt of Ag, FAD-Lactose-Salt of Co, FAD-Lactose-Salt of Zn, FAD-Lactose-Salt of Fe, FAD-Lactose-Salt of Ti are also part of the invention. FAD-Lactose-Salt of Au, and even more FAD-Lactose-Salt of HAuCl4*6H2O is preferred.

### Composition

A composition according to the invention comprises a pharmaceutically acceptable carrier and any of the foregoing compounds, preferably FAD-PEG-Diacid (more preferably FAD-PEG-600 diacid), FAD-Alginic Acid (FAD-ALG), FAD-Poly-Lactide, FAD-Bis-Phosphonate, FAD-Gelatine, FAD-Maltodextrin, FAD-Poly Amino Acid (FAD-Poly-L-Lysine), FAD-Poly-L-ornithine, FAD-Poly-L-arginine), FAD-lauryl-polyglucose, FAD-chitosan, FAD-collagen, preferably FAD-collagen I or FAD-human collagen IV or FAD-collagen I of a rabbit or FAD-collagen IV of rabbit, more preferably FAD-human collagen I or FAD-human collagen IV, and even more preferably FAD-human collagen I.

A fatty acid combined with FAD according to the invention is any one of the fatty acids selected from Oleic Acid, Myristic Acid, Nervonic Acid, Palmitic Acid, Linolenic Acid, Eicosapentaenoic Acid (EPA), Docosahexaenoic Acid (DHA), and a combination of these fatty acids.

Compounds FAD-Oleic Acid, FAD-Myristic Acid, FAD-Nervonic Acid, FAD-Palmitic Acid, FAD-Linolenic Acid, FAD-Eicosapentaenoic Acid, FAD-Docosahexaenoic Acid (DHA) are thus made according to the invention.

The Fatty Acids combined with any of the objects comprising FAD according to the invention described above are Oleic Acid, Myristic Acid, Nervonic Acid, Palmitic Acid, Linolenic Acid, Eicosapentaenoic Acid (EPA), Docosahexaenoic Acid (DHA), a combination of these fatty acids.

Individually, each of the above fatty acids is combined with the PEG diacid, to obtain a fatty acid combined with FAD-PEG or PEG-FAD, i.e. FAD-PEG-Oleic Acid, FAD-PEG-Myristic Acid, FAD-PEG-Nervonic Acid, FAD-PEG-Palmitic Acid, FAD-PEG-Linolenic Acid, FAD-PEG-Eicosapentaenoic acid, FAD-PEG-Docosahexaenoic acid, PEG-FAD-Oleic acid, PEG-FAD-Myristic acid, PEG-FAD-Nervonic acid, PEG-FAD-Palmitic acid, PEG-FAD-Linolenic acid, FPEG-FAD-Eicosapentaenoic acid, PEG-FAD-Docosahexaenoic acid.

### Liposome

Generally speaking, a liposome is an artificial vesicle formed by concentric lipidic bilayers, imprisoning aqueous compartments between them.

Liposomes include “small unilamellar vesicles or SUVs” with a size between 20 and 100 nm, “large unilamellar vesicles or LUVs” with a size between 100 and 1000 nm, and “giant unilamellar vesicles or GUVs” with a size greater than 1000 nm.

The oligolamellar vesicles “oligolamellar vesicles or OLV” have a size between 100 and 500 nm and have about 5 concentric bilayers. Multilamellar vesicles (MLVs) are multilamellar vesicles with a size greater than 500 nm and have several concentric bilayers (between 5 and 20). Multi-vesicular vesicles “multivesicular vesicles or MV V” are vesicles made up of several non-concentric bilayers trapped in a larger vesicle with a size greater than 1000 nm.

A liposome of FAD according to the invention is a particle formed by concentric lipid bilayers, enclosing between them aqueous compartments comprising an effective amount of FAD or an effective amount of any of the compounds comprising FAD according to the invention.

Two types of lyposome-FAD according to the invention are preferred:

A liposome comprising FAD-polyethylene glycol according to the invention is a liposome in which FAD-PEG is grafted onto phospholipids or cholesterol, for steric stabilization and increased residence time in the vascular system. The result is that metastasised or metastatic cancer cells can be reached and prevented from spreading.

A liposome comprising a FAD-polyethylene glycol grafted on phospholipids or cholesterol with an additional targeting agent makes it possible to concentrate the amount of liposome-PEG-FAD at the tumour level.

FAD is preferably at least partially encapsulated, i.e. at least part of the FAD is associated with the vector to form the particle.

According to a first variant, the FAD is at least partially encapsulated in a particle whose vector is a biopolymer or a mixture of biopolymers. Preferably, the vector is PEG. As an alternative, the vector is chitosan associated or not with glucose. The particle is a micelle that can be preferably micrometric or preferably nanometric in size. The micelle ensures encapsulation of the FAD in its core.

Advantageously, according to this first embodiment, the particle is formed by a manufacturing process such as forming of an emulsion by stirring the two compounds or by stirring in a supercritical fluid.

A second variant is that the FAD is at least partially encapsulated in a particle whose carrier is a biopolymer or a mixture of biopolymers and a metal. Preferentially, the metal is a nanoparticle, preferably of gold.

In addition to FAD's inherent anti-cancer action, complexes of FAD with gold have the property of reacting to infrared radiation to provoke intracellular hyperthermia that can be used therapeutically. The photothermal effect begins beyond a thermal elevation of more than 4° C. This is the case as illustrated in example 11 and FIG. 4. Thus, it is possible to destroy cancer cells, having absorbed a particle according to the invention comprising encapsulated FAD and at least one gold nanoparticle and a biopolymer as a vector, by means of a laser thermal probe with infrared radiation. The hyperthermia effect is added to the inhibitory effect of the FAD.

These gold nanoparticles are selected to ensure stable, easy to make, and highly reproducible nanoparticles with biocompatibility with the biomolecules that are to be grafted onto their surface or encapsulated.

Advantageously, the vector comprises PEG and at least one gold atom, preferably a gold nanoparticle.

The particle is formed by the FAD which is covalently bound such as a carbodiimide bond (EDC/NHS) to the PEG, the PEG being complexed with at least one gold nanoparticle.

According to a first embodiment, the PEG is complexed with at least one gold nanoparticle and the FAD is covalently bonded to the PEG partially at the surface of the particle. The particle according to this first embodiment is advantageously obtained by a manufacturing process called the ON method comprising a first step of particle synthesis, preferably of gold nanoparticles and PEG, then a second step of coupling of the FAD, in particular by carboiimide chemistry on the PEG. The first step of chemical synthesis involves mixing a gold Salt in the form of HAuCI4 with PEG.

Following a second embodiment, the FAD is complexed by a coordination reaction with at least one gold atom, preferably a gold nanoparticle, and covalently bonded to the PEG. The particle according to this second embodiment is advantageously obtained by a manufacturing process known as the IN method comprising a first step of complexing the FAD with at least one gold atom and then a second step of coupling the gold-FAD complex with the PEG. Advantageously, the first step preferably involves mixing a gold Salt in HAuCl4 form with the FAD to form the gold-FAD complex. The FAD is bound by a coordination bond to the gold atom. Advantageously, the second step preferably comprises mixing the gold-FAD complex with PEG. The PEG forms a polymeric network encapsulating the gold-FAD complex. A subsequent reduction step reduces the gold Salts to neutral gold atoms by adding for example NaBH4.

### METHOD FOR MANUFACTURING THE PARTICLES OR NANOCAPSULES ACCORDING TO THE INVENTION

The FAD in accordance with the invention may be produced by different methods such as the one described in U.S. Pat. Nos. 3,445,336, 4,255,566A, or FR1437964 incorporated here in full.

The present invention relates, in one aspect, to a process for manufacturing FAD particles, preferably microparticles of FAD and more preferably nanoparticles of FAD.

According to the invention, FAD alone or in the form of a particle, macroparticle, or nanoparticle can be encapsulated, preferably in a biopolymer and/or a phospholipid in the form of capsules, to produce FAD capsules, preferably FAD macrocapsules, and more preferably FAD nanocapsules.

According to a first aspect, the present invention relates to a process for manufacturing FAD particles, preferably FAD microparticles, and more preferably FAD nanoparticles, comprising:

a) a step of dispersion-mixing of two phases, by emulsion, of which at least one phase comprises FAD and at least one phase comprises a vector, (the phase comprising the FAD may be the same as that comprising the vector),

Optionally, the step is followed by a homogenisation step to stabilise the emulsion and obtain nanoparticles, or

a′) a step of a stirring in a supercritical fluid of a vector with FAD.

According to this first variant, a vector is a vector according to the invention preferably selected from a biopolymer or a mixture of biopolymers, a metal in the form of a salt, a lipid, preferably a phospholipid, or a combination of these vectors, preferably the vector is PEG or chitosan associated or not with glucose as a polymer.

The particle obtained according to the dispersion-mixing step is a micelle which can be micrometric, preferably nanometric.

The process according to this first variant optionally includes an additional encapsulation step, preferably encapsulation to form liposomes (capsules, macrocapsule, nanocapsule of phospholipids) comprising FAD, alone or associated with a vector.

### Salt-Polymer Followed by Fad

According to a second preferred variant, the present invention relates to a process for the manufacture of FAD particles, preferably FAD macroparticles, and more preferably FAD nanoparticles, in which process the FAD is associated with a vector comprising a mixture of biopolymers and a metal, preferably a vector in the form of a nanoparticle.

Preferably, the metal is a nanoparticle of metal more preferably, a gold nanoparticle.

In accordance with this second aspect, the present invention relates to a process for the manufacture of FAD particles, preferably FAD macroparticles, and more preferably FAD nanoparticles comprising:

i) a step of mixing a metal salt with a biopolymer, preferably the metal salt is a metal salt according to the invention, the biopolymer is a biopolymer according to the invention, even more preferably the metal salt is a gold salt in HAuCI4 form and the biopolymer is a PEG,

ii) a step of complexing FAD with the metal salt of the metal salt-biopolymer combination and/or

ii′) a step of binding the FAD with the biopolymer of the metal salt-biopolymer combination

iii) optionally a step of reduction of the metal salt to a neutral metal atom by addition of a reducing agent, e.g., NaBH4.

iii′) possibly a step of purification, preferably by centrifugation at 3×5000 rpm.

FAD-SALT, then Optionally POLYMER

According to a more preferred variant, the present invention relates to a process for the manufacture of FAD particles, preferably FAD macroparticles, and more preferably FAD nanoparticles comprising:

i) a step of mixing a metal salt with the FAD,

preferably a metal salt is a metal salt according to the invention, more preferably a metal salt in nanoparticle form and even more preferably a gold salt, in HAuCI4 and nanoparticle form. This step results in the manufacture of a FAD-metal salt particle, preferably a macro FAD-metal salt particle, and more preferably a nanoparticle of FAD-metal salt.

This step is possibly followed by a purification step for the nanoparticles of FAD-metal salt.

The manufacturing process of FAD particles may include a second step:

ii) a step of complexation s of the FAD-metal salt particle to a biopolymer, preferably a biopolymer according to the invention in the presence of a cross-linking agent such as EDC/NHS (1-ethyl-3-(3-dimethylaminopropyl)carbodiimide/N-hydroxysuccinimide), preferably the polymer is a biopolymer according to the invention, or a mixture of biopolymers according to the invention,

Optionally the process can still comprise,

iii) a step of reducing the metal salt into a neutral metal atom by the addition of a reducing agent, e.g. NaBH4. and/or

iv) optionally a purification step, preferably by centrifugation at 3× to 5000 rpm

The optional step of purification of nanoparticles metal salt FAD nanoparticles after the first step i) above or at iv) allows the manufacture of stable, easy to make and highly reproducible metal salt FAD nanoparticles with biocompatibility with the biomolecules with which they are grafted or encapsulated.

In a advantageous variant, the FAD-metal salt particle according to the invention is combined with a fatty acid, preferably a phospholipid, to form a liposome (capsule) comprising FAD-metal salt.

According to a favourable variant, the particle obtained by any one of these methods (FAD, metal salt, biopolymer) is associated with a fatty acid, preferably a phospholipid, to form a liposome (capsule) comprising the (FAD, metal salt, biopolymer).

The first step preferably involves mixing a gold salt in HAuCI4 form with the FAD to form the gold-FAD complex. The FAD is bound by a coordination bond to the gold atom. The second step preferably comprises mixing the gold-FAD complex with PEG. The PEG forms a polymeric network encapsulating the gold-FAD complex. A subsequent reduction step reduces the gold salts to neutral gold atoms by adding for example NaBH4.

Advantageously, the vector comprises PEG, according to the invention, and at least one gold atom, preferably a gold nanoparticle.

A particle is formed by the FAD which is covalently bonded, such as a carbodiimide bond (EDC/NHS) to the biopolymer, for example, PEG, the PEG being complexed with at least one gold nanoparticle.

According to an embodiment, the process according to the invention comprises of:

a first step of synthesizing a particle, preferably of gold and PEG nanoparticle, followed by the second step of FAD coupling, by carbodiimide chemistry on the PEG. The first step of chemical synthesis involves mixing a gold salt in the form of HAuCl4 with PEG.

Following a second embodiment, the FAD is complexed by a coordination reaction with at least one gold atom, preferably a gold nanoparticle, and covalently bonded to the PEG. The particle according to this second embodiment is advantageously obtained by a manufacturing process called the IN method comprising a first step of complexing the FAD with at least one gold atom and then a second step of coupling a gold-FAD complex with PEG. Advantageously, the first step comprises mixing a gold salt in HAuCl4 form with FAD to form the gold-FAD complex. The FAD being linked by coordination bond with the gold atom. Advantageously, the second step comprises mixing the gold-FAD complex with PEG. PEG forming a polymer network encapsulating the gold-FAD complex. A subsequent reduction step makes it possible to reduce the gold salts to a neutral gold atom by adding, for example, NaBH4.

According to a mode of realisation, the particle includes at least one type of targeting agent. Targeting agents aim to make the particle's penetration into cancer cells more specific. For example, the targeting agents are selected from peptides, monoclonal antibodies, aptamers, in particular, the HIV TAT-1 protein is a targeting agent advantageously used in the invention.

The targeting agents according to the invention are selected from the HIV Tat1 peptide, monoclonal or polyclonal antibodies, such as Kv-11 and Kv11, Aptamers, Anti-EGFR antibody, siRNA, Galectins, in particular, those selected from Gal-1, Gal-2, Gal-3, Gal-4, Gal-5, Gal-6 Gal-7; Interleukin-6 (IL-6), Superoxides dismutases (SOD) especially MnSOD, SOD2, SOD4, HIV Tat1 peptide is preferred.

Superoxide dismutases (SOD) are metalloproteins which are also oxidoreductases catalysing the dismutation of the superoxide 02 anions into oxygen 02 and hydrogen peroxide H2O2. The ones used according to the invention are Mn SOD, SOD2, and SOD4 (Hileman EA1, Achanta G, Huang P. Superoxide dismutase: an emerging target for cancer therapeutics. Expert Opin Ther Targets. 2001 December; 5(6):697-710).

Manganese superoxide dismutase (SOD1) is a dimeric protein found in the cytoplasm and mitochondrial interspace. The second type of CuZn-SOD (SOD3) is a tetrameric extracellular protein. This protein can bind to the surface of cell membranes or in type 1 collagen and protects cells from exogenous oxidative stress. Mn-SOD (SOD2) is located in the mitochondrial matrix but also on the inner wall of the mitochondria protecting them from the oxidative stress generated by the whole chain involved in cellular respiration.

The particle is advantageously made up of gold atoms, FAD molecules, and Polyethylene glycol (PEG), with or without the incorporation of a targeting agent.

According to one aspect, the invention concerns a composition, advantageously therapeutic, for use in the treatment and/or prevention of cancers, the composition comprising FAD, advantageously in a therapeutically effective quantity, and a therapeutically adapted vehicle.

The composition is intended to be used as the main active principle or as an adjuvant or neo-adjuvant of anti-cancer treatment.

This therapeutic composition may comprise a mixture, in variable proportions, of free FAD and FAD at least partially encapsulated in particle form. The composition comprises free FAD and particles as described above comprising a vector and FAD at least partially encapsulated by the vector. FAD is referred to as free because it is not bound to a particle.

This combination of two forms of FAD provides the first quantity of FAD very quickly thanks to the free form and then the encapsulated FAD releases over time the FAD ensuring a long-lasting administration.

The composition is advantageously formulated to be suitable for parenteral administration including intravenous, intramuscular, subcutaneous, and/or vaginal or rectal administration depending on the location of the cancerous lesions to be reached.

The composition is preferably formulated to be suitable for intravesical administration.

The composition is well formulated to be suitable for use as an injection solution, oral solution, and/or gel.

According to one aspect, the invention concerns a nanoformulation of FAD. The nanoformulation preferably comprises FAD at least preferentially encapsulated in or with a particle comprising a vector and FAD. Preferably, the nanoparticle has a diameter of less than 50 nm.

A preferred approach is that the effective plasma concentration of FAD is between 1 and 10 μM/L. In fact, the inhibitory concentration (IC50), which reduces the growth of cancer cells in culture by 50%, has been found in a range of 0.5 to 5 μM/L.

Typically, FAD, a FAD particle (micro or nanoparticle of FAD, FAD MNP) according to the invention or a pharmaceutical composition comprising FAD, or FAD MNP according to the invention, as described herein are administered to the subject in a therapeutically effective amount.

By “therapeutically effective amount” a FAD particle (micro or nanoparticle of FAD), FAD MNP according to the invention as described above, is meant a sufficient amount of FAD, FAD Micro or Nanoparticle, FAD MNP to attenuate, prevent, neutralize, treat or even eliminate cancer with a reasonable benefit/risk ratio applicable to any medical treatment.

The patient's risk in the case of the invention is lower and represents an advantage compared to the treatment previously proposed since no side effects are observed.

The patient's risk in the case of the invention is lower and represents an advantage compared to the treatment previously proposed since no side effects with FAD are observed.

Thus, the invention has another aspect: a combination of FAD and another anti-cancer drug, this other anti-cancer drug may be used at a lower dose than the dose usually used and induce an anti-cancer effect equivalent and/or greater than the sum of the anti-cancer effects added by the FAD and the said anti-cancer drug at the dose used, and therefore with fewer side effects than those observed with the same conventional anti-cancer drug used alone and at a dose inducing an equivalent anti-cancer effect.

The advantage of using FAD in combination with an anti-cancer agent is therefore to obtain an anti-cancer effect with little or no side effects.

However, it should be understood that the daily use of these compounds or objects according to the present invention will be decided by the treating physician in the context of an informed medical judgment.

The level of therapeutically effective dose specific to a particular subject will depend on various factors, including the disorder to be treated and the severity of the disorder; the specific activity of the FAD or FAD MNP particles used; the specific composition used, the age, body weight, general health, sex and diet of the subject; the timing, route of administration and rate of excretion of the specific FAD MNP and the combination used; the duration of treatment; and other drugs that may be used.

For example, It is quite classical to start treatment doses at lower levels than those necessary to achieve the desired therapeutic effect and to gradually increase the dose until the desired effect is achieved. In the present case, such precautions are not necessary since treatment with FAD has no detectable side effects up to doses of more than 1000 mg/kg.

However, the daily dosage of the FAD according to the invention or products derived from the FAD according to the invention, may vary from 0.01 to 1,000 mg of FAD per adult per day. The compositions typically contain 0.01 mg, 0.05, 0.1, 0.5, 1 mg, 0.1 g, 2.5, 5.0, 10.0, 15.0, 25.0, 50.0, 100, 250 and 500 g of FAD or particle of FAD (micro or nanoparticle of FAD) MNP, according to the invention, or a composition comprising FAD, particle of FAD (micro or nanoparticle of FAD MNP) according to the invention.

A medicament according to the invention contains from about 0.01 mg to about 50,000 mg (50 g) of FAD, preferably from 1 or 5 mg to about 100 to 500 mg of FAD. An effective amount of the medicine is provided at a dosage level ranging from about 0.0002 mg/kg to about 20 mg/kg body weight per day, in particular from about 0.001 mg/kg to about 7 mg/kg body weight per day. FAD, or one of its salts with a pharmacologically acceptable base or acid, is preferably used at a dose of and 0.1 mg or 50 g/kg, 3 to 10 mg/kg.

According to a particular embodiment, the FAD MNP or the composition comprising the FAD MNP according to the invention may be used at a concentration of between 0.01 μM and 20 μM, wherein, in particular, the FAD and the FAD MNP may be used at a concentration of 0.01, 0.05, 0.1, 0.5, 1.0, 2.5, 5.0, 10.0, 15.0, 20.0 μM.

According to the invention, the FAD or FAD MP or FADNP is administered to the subject in the form of a pharmaceutical composition.

The FAD and FAD MNP of the present invention can typically be combined with pharmaceutically acceptable excipients and optionally sustained-release matrices, such as biodegradable polymers, to form therapeutic compositions. “Pharmaceutically” or “pharmaceutically acceptable” refers to molecular entities and compositions which do not cause an adverse reaction, allergic or otherwise, when administered to a mammal, in particular a human being, as appropriate. Pharmaceutically acceptable carrier or excipient refers to a filler, diluent, encapsulating material or solid, semi-solid or liquid formulation auxiliary of any type.

In the pharmaceutical compositions of the present invention for oral, sublingual, subcutaneous, intramuscular, intravenous, transdermal, local or rectal administration, the active principle, alone or in association with another active principle, may be administered in a unitary form, mixed with conventional pharmaceutical carriers, to animals and human beings. Appropriate unit dosage forms include oral forms such as tablets, capsules, powders, granules and suspensions or oral solutions, sublingual and oral administration forms, aerosols, implants, subcutaneous, transdermal, topical, intraperitoneal, intramuscular, intravenous, transdermal. intrathecal and intranasal administration forms and rectal, intraurethral administration forms.

The pharmaceutical compositions according to the invention comprise FAD and vehicles which are pharmaceutically acceptable for a formulation capable of being administered orally, parenterally, intratumorally, intraperitoneally, subcutaneously, intramuscularly, intravenously, peros (it, ip sc, im, iv, po), or intraurethral, or intra vesicularly.

These may be, in particular, sterile, isotonic saline solutions (monosodium or disodium phosphate, sodium, potassium, calcium or magnesium chloride or similar, or mixtures of such salts), or dry compositions, in particular lyophilized, which, depending on the case, of sterilised water or physiological saline, allow the constitution of injectable solutions.

Pharmaceutical forms suitable for injectable use include sterile aqueous solutions or dispersions; formulations comprising sesame oil, peanut oil, or an aqueous solution of propylene glycol; and sterile powders for the extemporaneous preparation of sterile injectable solutions or dispersions. In any case, the form must be sterile and fluid as long as syringe facilities are available. It must be stable under manufacturing and storage conditions and must be preserved from the contaminating action of micro-organisms, such as bacteria and fungi.

Solutions comprising FAD or FAD-MP, FAD-NP in free base form, or pharmacologically acceptable salts may be prepared in water suitably mixed with a surfactant, such as a hydroxypropyl cellulose. Dispersions may also be prepared in glycerol, liquid polyethylene glycols, and mixtures thereof, and oils. Under normal conditions of storage and use, these preparations contain a preservative to prevent the growth of micro-organisms. A FAD or FAD MP or FAD NP and the combination of the present invention may be formulated in composition in neutral form or salt form.

Pharmaceutically acceptable salts include acid addition salts (formed with the free amino groups of the protein) and formed with inorganic acids such as, for example, hydrochloric or phosphoric acids, or organic acids such as acetic, oxalic, tartaric, mandelic acids, etc. The salts formed with free carboxyl groups can also be derived from inorganic bases such for example, sodium, potassium, ammonium, calcium, or ferric hydroxides, and organic bases such as isopropylamine, trimethylamine, histidine, procaine, and similar.

The manufacturing support for FAD particles can also be a solvent or dispersion medium containing, for example, water, ethanol, polyol (e.g. glycerol, propylene glycol, and liquid polyethylene glycol, etc.), their suitable mixtures, and vegetable oils. Appropriate fluidity can be maintained, for example, by using a coating, such as a lecithin, by maintaining the required particle size in the case of dispersion, and by using surfactants. Preventing the action of micro-organisms can be achieved by various antibacterial and antifungal agents, which are well known to the trade. In most cases, it will be preferable to include isotonic agents, for example, sugars or sodium chloride. Prolonged absorption of injectable compositions can be achieved by the use in the compositions of absorption retarding agents, e.g. aluminum monostearate and gelatine.

Sterile injectable solutions are prepared by incorporating the active compounds in the required quantity in the appropriate solvent with several of the other ingredients listed above, as required, followed by sterilisation by filtration.

Dispersions according to the invention are prepared by incorporating the sterilized agents of the present invention in a sterile vehicle which contains the basic dispersion medium and the other required ingredients from among those listed above. In the case of sterile powders for the preparation of sterile injectable solutions, typical preparation processes are vacuum drying and freeze-drying techniques which result in a powder of FAD or FAD MP or FAD NP and the combination of the present invention plus any additional desired ingredients from a sterile-filtered solution thereof.

More concentrated or highly concentrated solutions for direct injection are also being considered, as the use of DMSO as a solvent is assumed to result in extremely rapid penetration, delivering high concentrations of active agents to a small tumour area. The solutions will be administered in a manner compatible with the dosage formulation and a therapeutically effective amount. Formulations are easily administered in a variety of dosage forms, such as the type of injectable solutions described above, but drug delivery capsules and the like can also be used.

The invention relates in one aspect to a combination of FAD and at least one chemotherapeutic agent comprising:

(a) the FAD alone and/or the FAD according to the invention (in combination with at least one vector),

(b) at least one conventional chemotherapy drug.

The invention relates in one aspect to a combination of FAD and at least one chemotherapeutic agent comprising:

(a) the FAD according to the invention (alone and/or associated with a vector)

(b) at least one chemotherapeutic agent selected from
i) a Topoisomerase I inhibitor such as Irinotecan, topotecan
ii) a Topoisomerase II inhibitor—such as an anthracycline or an Epidodophyllotoxin (etoposide)
iii) a spindle poison such as a Taxane e.g. docetaxel, paclitaxel—a Periwinkle alkaloid such as vincristine, vinblastine, vindesine, vinorelbine,
iv) an antimetabolite—such as an antifolate like Methotrexate MTX, Pemetrexed—a purine analog-like fludarabine—a pyrimidine analog-like 5FU, gemcitabine, cytarabine,
v) an alkylating agent selected from nitrogen mustard, nitrosourea, alkylsulphonate, and sulfate (such as methyl triflate and dimethyl sulfate), a platinum salt such as trimethyloxonium tetrafluoroborate, cysplatinum, carboplatinum, oxaliplatinum, an oxazaphosphorine such as cyclophosphamide, ifosfamide.

The invention relates in one aspect to a combination of FAD and at least one chemotherapeutic agent comprising:

(a) the FAD alone and/or the FAD according to the invention (in combination with at least one vector),

(b) at least one chemotherapeutic agent selected from

i. an agent acting on microtubules; ii. an alkylating agent; iii. an anti-metabolite agent; iv. an intercalating agent; v. an inhibitor of topoisomerase I or II; such as camptothecin and its derivatives; vi. a compound targeting/reducing protein or lipid activity or protein or lipid phosphatase activity; vii. a proteasome inhibitor; viii. a protein kinase inhibitor; ix. an antibiotic.

FAD or FAD according to the invention can be used at an inactive dose by itself or at any dose, (since FAD can be administered at all the doses tested <(up to at least 50 mg/kg) without having any side effects) and is advantageously combined with one of these conventional chemotherapy drugs, even at a dose acting on less than 50% of patients.

Fluorouracil, or 5-fluorouracil (5FU), is a drug used in the treatment of cancer. It belongs to the class of anti-metabolite drugs, a subclass of pyrimidine analogs.

One aspect of the invention relates to the preventive or curative treatment of cancer in an individual in need of it, in particular a human being, comprising a combination of FAD and at least one chemotherapeutic agent.

Furthermore, the present invention relates to a kit or a commercial product comprising:

(a) a pharmaceutical formulation comprising the FAD alone and/or associated with at least one vector,

(b) a pharmaceutical formulation of one or more chemotherapeutic agents (a) and (b) for simultaneous, separate, or sequential use.

Furthermore, the present invention relates to a kit or a commercial product comprising:

(a) a pharmaceutical formulation comprising the FAD alone and/or associated with at least one vector,

(b) a pharmaceutical formulation of one or more chemotherapeutic agents

(a) and (b) for repeated and sequential use, where FAD may be administered before, during, or after, or during and after at least one other anti-cancer agent.

Furthermore, the present invention relates to a kit or a commercial product comprising:

(a) a pharmaceutical formulation comprising the FAD in combination with at least one vector, preferably FAD-PEG

(b) a pharmaceutical formulation of one or more chemotherapeutic agents

(a) and (b) for simultaneous, separate, or sequential use.

Furthermore, the present invention relates to a kit or a commercial product comprising:

(a) a pharmaceutical formulation comprising the FAD in combination with at least one vector, preferably FAD-PEG

(b) a pharmaceutical formulation of one or more chemotherapeutic agents

(a) and (b) for repeated and sequential use, where FAD may be administered before, during, or after, or during and after at least one other anti-cancer agent.

Compounding partners (a) and (b) may be administered together, one after the other or separately, in a combined unit dosage form or at least two separate unit dosage forms. The unit dosage form may also be a fixed combination.

FAD is advantageous when used in combination with other chemotherapeutic agents in a cancer patient. There are both synergistic and additive advantages, both for efficiency and safety. The therapeutic effects of combinations of chemotherapeutic agents with FAD can lead to a decrease in safe dosage ranges while maintaining high efficacy, of each component (or one of them) of the combination and/or a decrease in side effects, an improvement in cancer immunity.

The invention also relates to a process for the prevention or treatment of proliferative diseases and/or diseases associated with angiogenesis in a mammal, in particular, a human being with a combination of pharmaceutical agents which comprises:

(a) FAD or FAD-vector according to the invention; and

(b) at least one chemotherapeutic agent.

The invention furthermore concerns, according to a preferred embodiments, pharmaceutical compositions comprising:

(a) a therapeutically effective amount of FAD-PEG according to the invention;

(b) an antimetabolite, preferably a pyrimidine analog and more preferably 5FU,

(c) a pharmaceutically acceptable carrier.

Furthermore, the present invention concerns, according to a preferred embodiment, a kit or a commercial product comprising:

(a) a pharmaceutical formulation of FAD-PEG

(b) a pharmaceutical formulation of one or more chemotherapeutic agents;

(a) and (b) for simultaneous, concurrent, separate, or sequential use.

Furthermore, the present invention concerns, according to a preferred embodiment, a kit or a commercial product comprising:

(a) a pharmaceutical formulation of FAD-PEG

(b) a pharmaceutical formulation of one or more chemotherapeutic agents comprising an antimetabolite, preferably a pyrimidine analog and more preferably 5FU,

(a) and (b) for simultaneous, concurrent, separate, or sequential use.

Standard chemotherapeutic agents combined with FAD or FAD, vector according to the invention.

The term “conventional chemotherapeutic agents” is broad and encompasses many chemotherapeutic agents with different mechanisms of action. The combination of these with FAD improves the treatment of cancer. As a general rule, chemotherapeutic agents are classified according to their mechanism of action.

The term “chemotherapeutic agent” refers in particular to any chemotherapeutic agent for cancer. This includes, but is not limited to, one or more of the following: i. an active agent on microtubules; ii. an alkylating agent; iii. an anti-metabolite agent; iv. an intercalating agent; v. an inhibitor of topoisomerase I or II; such as camptothecin and its derivatives; vi. an anti-inflammatory agent. a compound targeting/reducing protein or lipid activity or protein or lipid phosphatase activity; vii. a monoclonal antibody; viii. a proteasome inhibitor; ix. a protein kinase inhibitor; an antibiotic.

The term “microtubule active agent”, or “spindle poison” as used here, refers to microtubule stabilising agents, microtubule stabilisers, and microtubule polymerisation inhibitors, including, without limitation, taxanes, e.g. paciltaxel and docetaxel; vinca-alkaloids, e.g. vinblastine, in particular, vinblastine sulfate; vincristine, in particular, vincristine sulphate and vinorelbine; discodermolides; cochicine and epothilones and derivatives thereof, e.g. epothilone B or a derivative thereof.

Paclitaxel is marketed as TAXOL; docetaxel as TAXOTERE; vinblastine sulfate as VINBLASTIN R.P; and vincristine sulfate as FARMISTIN. Generic forms of paclitaxel are also included, as well as various galenic forms of paclitaxel. Generic forms of paclitaxel include but are not limited to, betaxolol hydrochloride. Various dosage forms of paclitaxel include, but are not limited to, paclitaxel, an albumin nanoparticle, marketed as ABRAXANE; ONXOL, CYTOTAX. Discodermolide can be obtained, for example, as described in U.S. Pat. No. 5,010,099.

Besides, the epotholine derivatives which are described in U.S. Pat. No. 6,194,181, WO 98/10121, WO 98/25929, WO 98/08849, WO 99/43653, WO 98/22461, and WO 00/31247 are also included; epotholine A and/or B in particular.

The preferred spindle poisons combined with FAD or FAD according to the invention,

vincristine, vinblastine, vinorelbine, vindesine, vinorelbine, vinflumine; ii) dolastatin, such as romidepsine; Paclitaxel, nab, paclitaxel, docetaxel, carbitaxel; epothilone, Ixabepilone, are particularly suitable.

The most preferred spindle poisons in combination with FAD or FAD, ocetaxel, paclitaxel—vincristine, vinblastine, vindesine, vinorelbine are particularly suitable.

A more preferred spindle poison in combination with FAD or FAD according to the invention is selected from Taxol, Docetaxel, Paclitaxel, Vincristine, Vinorelbine, Vindesine, and Vinblastine (Velbe).

The spindle poisons selected from Taxol, Taxotere (docetaxel), Paclitaxel, Vincristine are the most preferred in combination with FAD or FAD according to the invention.

Eribulin (Halaven) is a microtubule inhibitor. It is a synthetic analog with a simplified structure of halichondrin B, a substance isolated from the marine sponge Haichondria okadai.

Eribulin in combination with FAD or with FAD according to the invention is an object of the present invention.

The term “alkylating agent”, as used herein, includes, but is not limited to,

i) Nitrogenous mustards (melphalan, chlorambucil, estramutine; ii) Oxazaphosphorines, cyclophosphamide, ifosfamide; iii) Triazenes and hydrazines, procarbazine, dacarbazine, temozolomide; iv) Ethylene imines, aziridines, thiotepa, mitomycin C; v) Nitrosoureas BCNU, CCNU, fotemustin, streptozotocin; vi) Alkyl alkanes sulphonate, busulfan; vii) Organoplatins or “platinum compound”.

The term “platinum compound” as used herein includes, but is not limited to, carboplatin, cisplatin, cisplatin, oxaliplatin, satraplatin, and platinum agents such as ZD0473. Carboplatin may be administered, for example, in the form in which it is marketed, e.g., CARBOPLATE; and oxaliplatin as ELOXATIN.

Trabectedine is also combined with FAD or FAD according to the invention.

An intercalant selected from Melphalan, chlorambucil—BCNU—a platinum salt—an aziridine: mitomycin C can be combined with FAD or FAD according to the invention.

Within the intercalants, an anthracycline such as Doxorubicin ADRIBLASTINE® Epi or Adriamycin FARMORUBICINE® or Idarubicin ZAVEDOS® is particularly well adapted for use in combination with FAD or FAD according to the invention.

According to a preferred embodiment, FAD or FAD according to the invention is combined with any of the intercalants selected from cyclophosphamide, ifosfamide, Imelphalan, BCNU (or Gliadel), temozolamide (TEMODAR), Cisplatinum, Carboplatinum, free platinum or in salt form, Bendamustine, and Temodal.

Cyclophosphamide can be administered, with FAD or FAD according to the invention, for example, in the marketed form CYCLOSTIN; and ifosfamide as HOLOXAN.

According to a preferred embodiment FAD or FAD according to the invention is combined with Cyclophosphamide (endoxan).

According to a preferred mode of realization, FAD or FAD according to the invention is combined with Bendamustine.

According to a preferred mode of realization, FAD or FAD according to the invention is combined with Temodal.

Anti-metabolites interfere with DNA synthesis; they are structural analogs, on the one hand, of purine and pyrimidine bases (or corresponding nucleosides) and, on the other hand, of folinic coenzymes, as the latter are involved in numerous stages of purine and pyrimidine biosynthesis.

The term “anti-metabolite” or “anti-neoplastic anti-metabolite” includes, but is not limited to, 5-fluorouracil (5-FU); capecitabine; gemcitabine; DNA demethylation agents such as 5-azacytidine and decitabine; methotrexate; edatrexate; and folic acid antagonists such as, but not limited to, pemetrexed. Capecitabine may be administered, for example, in the form in which it is marketed, for example, under the brand name XELODA; and gemcitabine in the form of GEMZAR.

Anti-metabolites interfere with DNA synthesis of constituents; they are structural analogs, on the one hand, of purine and pyrimidine bases (or corresponding nucleosides) and, on the other hand, of folinic coenzymes, as the latter are involved in numerous stages of purine and pyrimidine biosynthesis. Their action is to inhibit cell replication, e.g. by incorporation into nucleic acids, which leads to cell death including DNA breakdown.

The preferred antimetabolites that may be associated with FAD according to the invention include,

i) Pyrimidine analogs such as 5-FU, tegafur, capecitabine, azacitidine, gemcitabine;

ii) Analogues of purines such as mercaptopurine, fludarabine, azathioprine, cladribine, pentostatin, cytarabine, nelarabine, clofarabine.

iii) Folic acid analogs such as methotrexate, pemetrexed, pralatrexate, ralitrexed, trimetrexate, piritrexine

iv) decitabine, sapacitabine are suitable.

An antimetabolite associated with FAD or FAD according to the invention is selected from 5-FU, Cytarabine, Capecitabine (xeloda), Fluoropyrimidine (Alimta), pemetrexed, Gemcitabine (gemzar), Tomudex (raltitrexed), more preferably 5-FU.

FAD, preferably FAD-PEG, and more preferably FAD-PEG600 is combined with an antifolate, methotrexate, or pemetrexed—with a purine analog, fludarabine, or a pyrimidine analog, 5FU. FAD, preferably FAD-PEG, and more preferably FAD-PEG600 is combined with 5-FU.

An appropriate dose of, e.g. 5-FU in humans is an appropriate dose in the range of 100-1500 mg per day, e.g. 200-1000 mg/day, such as 200, 400, 500, 600, 800, 900, or 1000 mg/day, administered in one or two doses per day. 5-FU can be administered to a human being in a dosage range of approximately 50-1000 mg/m2/day, e.g. 500 mg/m2/day.

Such doses may be reduced by at least 50% in combination with FAD according to the invention or the FAD.

Olaparib acts by inhibiting poly (ADP-ribose) polymerases (PARP). FAD, preferably FAD-PEG, and more preferably FAD-PEG600 is combined with Olaparib.

The term “proteasome inhibitor”, as used here, includes compounds that target, decrease or inhibit the activity of the proteosome. Compounds that target, reduce, or inhibit proteosome activity include, but are not limited to PS-341; MLN 341, bortezomib, or velcade.

FAD or FAD, according to the invention, is combined with bortezomib.

The term “topoisomerase I inhibitor”, as used herein, includes Irinotecan, topotecan, camptothecin, and its active derivatives.

Camptothecin acts by interfering with the unfolding of DNA super wound by the cellular enzyme topoisomerase I, which triggers events leading to apoptosis and programmed death in malignant cells.

The topoisomerase I inhibitor Campto (irinotecan) in combination with FAD according to the invention or with FAD is a pharmaceutical combination or a kit according to the invention.

The term “topoisomerase II inhibitor” as used here includes, but is not limited to, Anthracyclines (intercalants), doxorubicin, daunorubicin, epirubicin, idarubicin, and nemorubicin; Anthracenediones, anthraquinones, mitoxantrone, and losoxantrone; epidophyllotoxins, etoposide, teniposide; or amsacrine or even bleomycin; Epidodophyllotoxins (etoposide).

Etoposide has been marketed as ETOPOPHOS; teniposide as VM26-BRISTOL; doxorubicin as ADRIBLASTIN or ADRIAMYCIN; epirubicin as FARMORUBICIN; idarubicin as ZAVEDOS; and mitoxantrone as NOVANTRON. The daunorubicin, includes the liposomal formulation, e.g. DAUNOSOME;

Doxorubicin is preferred in combination with FAD or FAD according to the invention. Doxorubicin includes the liposomal formulation, e.g. CAELYX;

Epirubicin is preferred in combination with FAD or FAD according to the invention.

Etoposide, marketed as ETOPOPHOS, is preferred in combination with FAD or FAD according to the invention.

A topoisomerase II inhibitor selected from Etopophos (etoposide), topotecan, innotecan in combination with FAD according to the invention is a pharmaceutical combination or a kit according to the invention.

A topoisomerase II inhibitor selected from Etopophos (etoposide), topotecan, innotecan in combination with FAD is a pharmaceutical combination or a kit according to the invention.

Bricatinib is an inhibitor of ALK (“Anaplastic lymphoma kinase”) and EGF receptor;

Bricatinib and FAD or Bricatinib and FAD, according to the invention, constitute a pharmaceutical combination or a kit, according to the invention.

Palbociclib (Ibrance) is a molecule that inhibits two cyclin-dependent kinases, CDK4 and CDK6, and are proteins necessary for the cell cycle.

Palbociclib and FAD or Palbociclib and FAD according to the invention, constitute a pharmaceutical combination or a kit according to the invention.

Sorafenib (Nexavar) is a tyrosine kinase inhibitor. FAD and Sorafenib or Sorafenib and FAD according to the invention, constitute a pharmaceutical combination or a kit according to the invention.

The preferred classical chemotherapy drugs (b) for combination with FAD or with FAD according to the invention (a) are selected from any of the following classical chemotherapy drugs: Taxol, Taxotere (docetaxel), Paclitaxel, 5-FU, Etopophos (etoposide), Doxorubicin, Vincristine, Capecitabine (xeloda), Vinblastine (Velbe), Cyclophosphamide (endoxan), Alimta (pemetrexed), Cytarabine, Cisplatin, Carboplatin, Free Platinum, Olaparib, Epirubicin, Campto (innotecan), Gemcitabine (gemzar), Bricatinib, Eribulin (Halaven), Bortezomib (Velcade), Bendamustine, Palbociclib (Ibrance), Temodal, Sorafenib (Nexavar), Tomudex (raltitrexed), Letrozole (anti-aromatase, complementary hormone therapy);

According to another embodiment, the pharmaceutical composition of the invention concerns a combined preparation for simultaneous, separate, or sequential use in a process for inducing non-apoptotic signaling of a cancer cell in a subject suffering from cancer.

According to a particular mode of realization, the usual doses (prescribed by a physician) of conventional anti-cancer chemotherapy drug can be increased, and the side effects greatly reduced when the conventional anti-cancer chemotherapy drug is combined with FAD according to the invention (compared to the side effects observed without FAD or FAD according to the invention).

According to a particular mode of realization, the usual doses (prescribed by a physician) of conventional anti-cancer chemotherapy drug can be unchanged, and the side effects greatly reduced when the conventional anti-cancer chemotherapy drug is combined with FAD according to the invention (compared to the side effects observed without FAD or FAD according to the invention).

According to a particular embodiment, the usual doses (prescribed by a doctor) of conventional cancer chemotherapy drugs can be reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% when the conventional cancer chemotherapy drug is combined with the FAD according to the invention; the anticancer activity of the combination with the FAD is equivalent or superior to that measured with 100% of the active dose of the conventional anti-cancer chemotherapy drug.

The combination of the invention may also be applied in combination with other treatments, for example, surgery, hyperthermia, and/or radiation therapy.

Pharmaceutical compositions comprise about 0.00002% to about 100%, in particular, for example, in the case of ready-to-use infusion dilutions) from 0.0001 to 0.02%, or, for example, in the case of infusion or infusion concentrates, in particular parenteral. formulations, from about 0.1% to about 95%, preferably from about 1% to about 90%, more preferably from about 20% to about 60%, of active ingredient (by weight, in each case).

The components of the pharmaceutical compositions according to the invention may be, for example, in the form of a unit dose, such as in the form of ampoules, vials, tablets, infusion bags, or capsules.

For parenteral administration in an aqueous solution, for example, the solution must be appropriately buffered if necessary and the liquid diluent must first be made isotonic with a sufficient amount of saline or glucose. These particular aqueous solutions are particularly suitable for intravenous, intramuscular, subcutaneous transurethral, and intraperitoneal administration. In this respect, the sterile aqueous media which can be used will be known to the skilled person in the light of this description. Certain variations in dosage will necessarily occur depending on the condition of the subject being treated. In all cases, the person responsible for administration will determine the appropriate dose for each subject.

### EXAMPLES

For these examples below, the FAD molecule was obtained from Alfa Aesar.

The measurements were made on a UV-visible spectrometer Uvikon 941 Kontron instruments driven by Thermalys Uvikon 900 software. The solutions were placed in quartz cuvettes with a 1 cm optical path. The absorption spectra of the gold nanoparticles were recorded in the spectral range from 200 nm to 900 nm.

The Raman Spectrometry/SERS experiments were conducted with the Xplora spectrometer developed by Horiba Jobin Yvon. This spectrometer uses a monochromatic laser source, which is focused on the sample. The Raman signal scattered by the sample is collected with the same objective (backscatter configuration). The Raman signal is then directed to a diffraction grating, the image of which is collected by a CCD camera, resulting in a spectrum.

The study of the particles was carried out using the following parameters:
Excitation wavelength: 660 nm
Confocal collection hole: 300 μm
Diffraction network: 600 lines/mm, giving access to a spectral resolution of the order of 3 cm-1.
Acquisition time: 120 seconds repeated twice.

### Procedure According to the Invention

Preparation of the Polymer Solution:

10 mg of polymer, preferably PEG-600 diacid or ALG (from Alginate) are dissolved in PBS (10 mL PBS) buffered at pH=9.0 and of ionic concentration corresponding to NaCl: 0.5 mM. The solution is kept under stirring for one hour at room temperature (20° C.) before use.

Preparation of the Collagen Solution:

A Collagen solution (1 mg/mL) (Human Collagen 1 and IV or Rabbit Collagen 1 and IV, preferably Human Collagen 1 and IV, and even more preferably Human Collagen 1) is diluted in a solution of phosphate saline buffer (PBS) at pH=7.2, with an ionic concentration corresponding to NaCl: 0.5 mM. The solution is kept under stirring for one hour at room temperature (20° C.) before use.

Preparation of the Fatty Acid Solution:

A fatty acid, e.g. 10 mg Chitosan (CHIT), is dissolved in PBS (10 mL PBS for 10 mg CHIT) buffered at pH=9.0 of ionic strength corresponding to a NaCl concentration of 0.5 mM by stirring for 1 hour at 20° C. until the solution is homogenized.

Preparation of the Metal Salt Solution:

The metal salt (e.g. of HAuCl4*6H2O) is dissolved in purified water, e.g. 16 mg of metal salt (e.g. of HAuCl4*6H2O) is dissolved in 50 mL of purified water (MilliQ water).

### Procedures for the Synthesis of FAD Particles

Synthesis of FAD-Polymer (FAD-PEG)

40 mg of FAD are dissolved in 10 mL of extra pure water by stirring for 10 min. Then 250 μl (1 mM) of Polymer (e.g. Polyethylene glycol 600 Diacid) (PEG) are added to the solution. The resulting solution is purified by centrifuging, 3 times, at 5000 rpm for 5 min.

Reverse Synthesis of Polymer-FAD (PEG-FAD)

10 ml of a 1 mM aqueous polymer solution (e.g. Polyethylene glycol 600 Diacid (PEG)) is kept under stirring for 10 min. Then 10 mL of a FAD solution (40 mg/10 mL) is added and the mixture is kept under agitation for 30 min at 20° C. The resulting solution is purified by centrifugation, 3 times, at 5000 rpm for 5 min.

Synthesis of FAD-Metal Salt or FAD-Metal Salt of Au.

10 mg of FAD was dissolved in 5 mL of ultrapure water under agitation for 10 min. Then 20 ml (16 mg/50 ml water) of metal salt (e.g. HAuCl4*6H2O) were added and the mixture was kept under stirring for 30 min at 20° C. The resulting solution is purified by centrifugation, 3 times, at 5000 rpm for 5 min.

Synthesis of FAD-Metal Salt

20 ml of a metal salt solution (at 16 mg/50 ml of water) e.g. 20 mL of HAuCl4*6H2O are kept under stirring for 10 min at 20° C. Then 10 mg of FAD dissolved in 5 mL of ultrapure water are added and kept under stirring for 30 min at 20° C.

The resulting solution is purified by centrifugation, 3 times, at 5000 rpm for 5 min.

Synthesis of FAD-Metal Salt-Polymer

10 mg of FAD are dissolved in 5 mL of ultrapure water by magnetic stirring for 10 min. Then 20 mL of a metal salt solution (e.g. HAuCl4*6H2O) is added and the solution is kept under magnetic stirring for 20 min at 20° C. Then 250 μl (1 mM) of Polymer (e.g. Polyethylene glycol 600 Diacid (PEG)) is added and the mixture is stirred for 30 min at 20° C. The resulting solution is purified by centrifugation, 3 times, at 5000 rpm for 5 min.

Synthesis of Metal Salts-FAD-Polymer—(Au-FAD-PEG Salt)

20 mL of a 16 mg/50 ml solution of metal salt water (e.g. Au salt of formula HAuCl4*6H2O) are kept under stirring for 10 min at 20° C. Then 10 mg of FAD dissolved in 5 mL of ultrapure water are added and kept under agitation for 30 min at 20° C. Then 250 μl (1 mM) of polymer (such as Polyethylene glycol 600 Diacid (PEG)) is added and the mixture is stirred for 30 min at 20° C. The resulting solution is purified by centrifuging, 3 times, at 5000 rpm for 5 min.

Synthesis of Polymer-Metal-Salt FAD (Poly-Au-FAD)

10 ml of a 1 mM aqueous solution of Polymer (such as a solution of Polyethylene glycol 600 Diacid (PEG)) are kept under agitation for 10 min. Then 20 ml of a metal salt solution (16 mg/50 ml-water) as a solution of HAuCl4*6H2O) are added under stirring for 10 min at room temperature. Then 10 mL of a 40 mg/10 mL solution of FAD are added and kept under agitation for 30 minutes at 20° C.

The resulting solution is purified by centrifuging, 3 times, at 5000 rpm for 5 min.

Synthesis of Polymer-FAD-Metal Salt

10 mL of a 1 mM aqueous solution of Polymer (such as a 1 mM aqueous solution of Polyethylene glycol 600 Diacid (PEG)) are kept under agitation for 10 min. Then 10 mL of a 40 mg/10 mL FAD solution is added with agitation for 30 min at 20° C. Then 20 ml of a 16 mg metal salt solution/50 ml water (e.g. 20 mL of a 16 mg HAuCl4*6H2O solution/50 ml water) are added and stirred for 10 min at 20° C.

The resulting solution is purified by centrifuging, 3 times, at 5000 rpm for 5 min.

Synthesis of Nanoparticles of FAD-Metal Salts (FAD@AuNPs)

20 ml of a 0.0001M aqueous solution of metal salt (HAuCl4 6H2O) are kept under stirring for 10 min. Then 10 ml of FAD is added and stirred for 10 minutes. Then 250 μl of a polymer solution at a concentration of (1 mM) (Polyethylene glycol 600 Diacid (PEG) etc.) are added and mixed for 20 min at 20° C./After 10 min, 3 mL of NaBH4 (0.01M) are added drop by drop and mixed vigorously by stirring for 2 hours. The resulting pinkish-red solution is purified by centrifugation, 3 times, at 5000 rpm for 5 min.

The synthesis of FAD-AuNP nanoparticles was also carried out under the same conditions as those described for FAD-polymer or metal salt-FAD-Polymer complexes in the presence of a reducing agent (NaBH4).

Liposomes comprising FAD, FAD-PEG, FAD-ALG, FAD-Collagen I, FAD-CHITOD+SAN, or FAD-PEG-Au are obtained according to the invention. Liposomes are prepared, for example, as described in (Akbarzadeh, A., Rezaei-Sadabady, R., and others (2013). Liposome: category, preparation, and applications. Research letters of Nanoscale 8(1). DOI: 10.1186/1556-276X-8-102., or Li, M., du, C., Guo, N., Teng, Y., Meng, X., Sun, H., Li, S., Yu, P., and Galons, H. (2019). Composition model and medical application of liposomes. European Tourillon of Medicinal Chemistry 164; 640-653. DOI: 10.1016/j.ejmech.2019.01.007) obtained from these preparations.

Other preparations comprising FAD only, or FAD-PEG, or FAD-Au Salt have been made according to a method described in:

J. Rosselgong, M. Chemin, C. Cabrai Almada, G. Hemery, J-M. Guigner, G. Chollet, G. Labat, D. da Silva Perez, F. Ham-Pichavant, E. Grau, S. Grelier, S. Lecommandoux, H. Cramail

Synthesis and self-assembly of Xylan-based amphiphiles: from bio-based vesicles to antifungal properties

ACS Biomacromolecules—October 2018

DOI: 10.1021/acs.biomac.8b01210

### Example 1

Procedure for synthesis of gold-FAD-PEG particles by an IN method.

The complex is mainly prepared with gold salts, typically chlorauric acid (HAuCl4, Aldrich) with a concentration of 1 mM. After dissolving the gold salt, the solution is stirred vigorously and the FAD solution is added (5 ml at a concentration of 40 μM), after a few moments 250 μL, of PEG and 600 μL, of NaBH4 are added. The chemical bonding will be done by coordination at the phosphate groups of the ribose or ketone groups of the PEG flavin pattern. The FAD is encapsulated within the core of the PEG particle.

The synthesis procedure is shown in FIG. 1.

Purification of the particle is done by ultracentrifugation at 9000 rpm. The solution is centrifuged at 9000 rpm, the supernatant is removed, the pellet is resuspended in water, these three steps are repeated 3 times in a row.

The particle is preferably purified by ultracentrifugation at 5000 rpm. The solution is centrifuged at 5000 rpm, the supernatant is removed, the pellet is resuspended in water, these three steps are repeated 3 times in a row.

### Example 2

Procedure for synthesis of gold-FAD-PEG particles by an ON method.

Colloidal gold solutions are prepared by reduction of gold salts, typically chloraluric acid (HAuCl4, Aldrich) with a concentration of 1 mM. After the dissolution of the gold salt, the solution is stirred vigorously and the reducing agent NaBH4 (Sodium tetrahydridoborate, Sigma Aldrich) is added, reducing the Au3+ ions to neutral gold atoms (AuO). During the reaction, more and more gold atoms are produced, the solution becomes supersaturated and the gold atoms begin to precipitate as subnanometric particles.

To prevent the particles from aggregating with each other, a stabilizing agent is advantageously added, PEG-COOH (Poly-Ethylene glycol dicarboxylic acid, Sigma Aldrich), at room temperature. The PEG molecules, thanks to their dual hydrophobic and hydrophilic nature, can interact with an AuCl-cluster and produce, thanks to a reduction process, well-dispersed gold nanoparticles with a size of about 10 nm.

The FAD biomolecule is grafted by Carbodiimide bonds using the EDC/NHS activator couple (for 1-Ethyl-3-(3-dimethylaminopropyl)-carbodiimide/N-hydroxysuccinimide) (40 mg/10 mg). The activating couple will activate the COOH group of the PEG, and the NH2 group of the FAD, creating a covalent Carbodiimide bond.

FAD is added in a quantity of 500 μL, to this solution with 4500 μL of gold nanoparticles, making a concentration of 40 μM FAD in the solution.

Purification of the particle is carried out in the same way as described in example 1. The FAD is complexed with a gold nanoparticle. The FAD is carbodiimide bonded to the PEG. The FAD is arranged on the surface of the gold nanoparticle-PEG.

According to the invention the FAD metal salt ratio can be 0.05:1, 0.5:1, 1:1, 2:1, 3:1, 4:1, 5:1, 10:1.

According to the invention the biopolymer ratio: FAD can be 0.05:1, 0.5:1, 1:1, 2:1, 3:1, 4:1, 5:1, 10:1.

### Example 3

Characterization of gold-FAD-PEG particles synthesized by an IN method.

The particles obtained according to example 1 are characterized by the measurement of UV-visible absorbance allowing the coupling of the particles with the FAD to be seen.

The results show UV-visible spectra of the formation steps of the nano-sized particles, i.e. nanoparticles:
a first spectrum corresponds to the gold salt-FAD complexes, we can just observe the signature of the FAD, which corresponds to the complexation of the FAD to the gold salts,
a second spectrum corresponds to the gold salts-FAD-PEG, it can be seen that no nanoparticles have been formed, the gold salts are not reduced, there is no plasmon band,
a spectrum corresponds to the particle Gold-FAD-PEG-reducing agent NaBH4, a plasmon band can be observed at 560 nm, which corresponds to the formation of nanoparticles. A colloidal solution stabilized by the presence of PEG is obtained.

The samples are passed through the MET (Transmission Electron Microscopy), which allows the formation of nanoparticles to be confirmed.

The particles obtained according to example 1 are characterized by SERS allowing us to see the coupling of the nanoparticles with the FAD.

The unrepresented results show a first spectrum representing the formation of the particles showing peaks corresponding to the FAD powder (second spectrum) allowing to see the formation of particles.

### Example 4

Characterization of gold-FAD-PEG particles synthesized by an ON method.

The gold-PEG-FAD particles obtained according to example 2 are characterised by the UV-visible absorbance measurement, which shows the coupling of the nanoparticles with the FAD.

The particle alone, before and after FAD functionalization are compared with the negative inspection which is FAD alone in powder form.

In the spectrum after FAD functionality, the plasmon band widens, shifts red at 530 nm, and becomes asymmetric. This asymmetry appears to correspond to the adhesion of the FAD to the surface of the nanoparticles.

The samples are passed through the MET (Transmission Electron Microscopy), which allows the formation of nanoparticles to be confirmed.

The particles obtained according to example 2 are characterised by SERS allowing us to see the coupling of the nanoparticles with the FAD.

Three non-represented SERS spectra are obtained: that of gold nanoparticles alone, that of gold nanoparticles after covalent grafting of FAD, and that of the FAD molecule in powder form.

In all cases, there is a band at 1640 cm-1 which corresponds to water, the solvent for the nanoparticles. Besides, Raman bands are observed which correspond to the signature of the PEG-COOH which coats the nanoparticles: bands at 1137 cm-1 due to the vibration of the COC group, at 1270 cm-1 which corresponds to the vibration of the COH group, and at 1455 cm-1 corresponding to the vibration of the CO group.

The spectrum of FAD grafted by covalent bonding still shows the peaks of the PEG-COOH, but a new peak appears at 1349 cm-1 for the adenine, and at 1462 cm-1 for the C—O group. On the spectrum representing FAD in powder form, several peaks at 1349 cm-1 and 1462 cm-1 correspond to FAD. This FAD spectrum is taken as a reference to see the FAD signature.

### Example 5

Characterization of FAD release by gold FAD-PEG particles synthesized by an IN method.

The particles obtained in example 1 are centrifuged at 13000 rpm, for 30 min at 4° C., the pellet is recovered and resuspended in other media including PBS pH 7 and 5.

The samples are incubated at 37° C. for different durations of 1 h, 5 h, 24 h, 48 h, and 72 h to follow the evolution of the release by extinction spectroscopy (UV-visible) and vibrational spectroscopy (SERS).

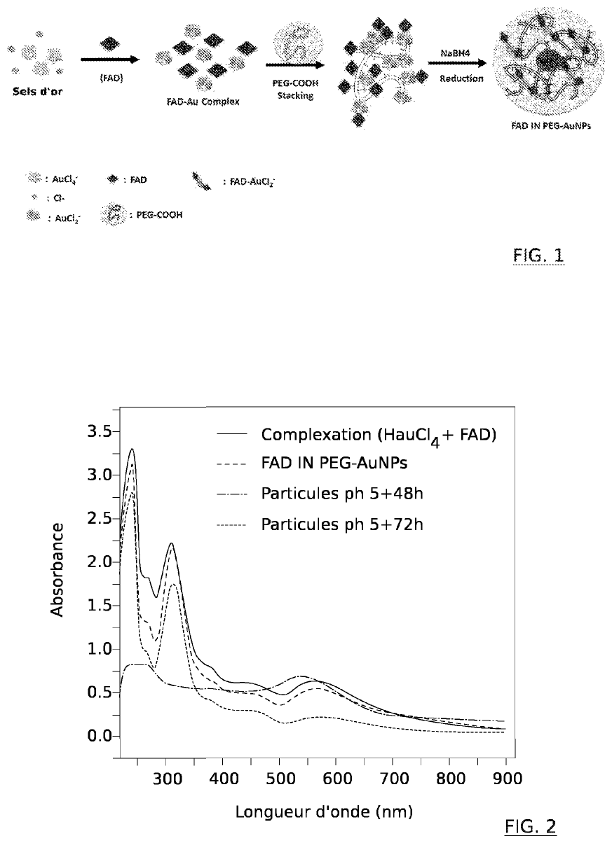

The results of the UV-visible spectra (FIG. 2) show the release of FAD as a function of time at pH 5. It is more than 90% at 24 H

In the spectrum by SERS before the release (not shown) Raman bands are observed which correspond to the signature of the grafted FAD inside the complex. After having changed the nanoparticle medium (PBS pH 5 and 7), the disappearance of the peaks that signal the presence of FAD is observed in a concordant manner. This corresponds to the release of FAD with time.

### Example 6

Protection of FAD in different particles.

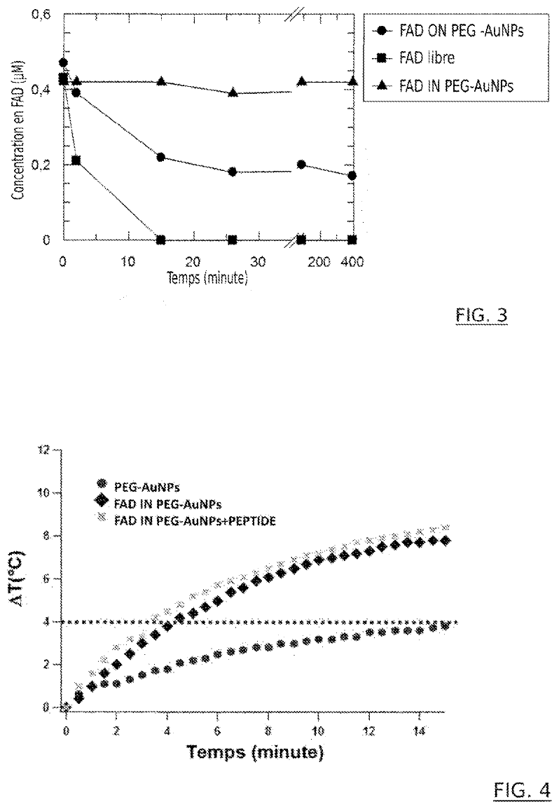

FAD protection in particles in comparison with unbound FAD is studied. The results are shown in FIG. 3.

Different particles containing FAD are compared to unbound FAD:
The FAD encapsulated with a gold FAD-PEG particle ON is obtained by the ON method described in example 2.
The FAD encapsulated with a gold-FAD-PEG particle IN is obtained by the IN method obtained in example 1.

The different particles and the unbound FAD are each placed in a solution of the Nucleo PyroPhosphatase enzyme at 25° C. The concentration of the FAD is monitored over time by HPLC assay.

It is shown that the free FAD is fully hydrolyzed within 15 minutes when the concentration of FAD encapsulated by the IN method is unchanged after 400 minutes. The FAD encapsulated by the ON method shows hydrolysis of almost 50% of the FAD at 400 minutes.

### Example 7

PEG-FAD particle synthesis, characterisation, and stability. 5 ml of FAD at 80 μM is mixed with 500 μl of PEG, under continuous magnetic stirring for about 4 hours.

PEG-FAD particles are obtained. A purification step of the particles is performed as described in example 1.

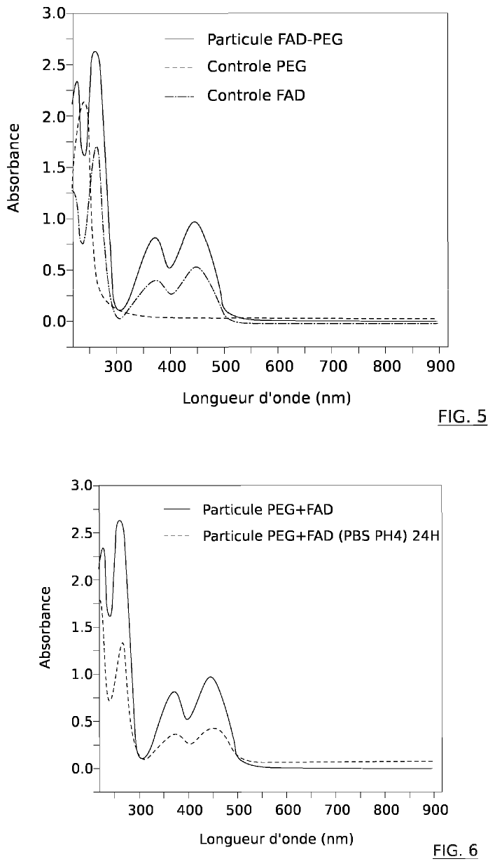

The characterisation of the particle by UV-visible (extinction spectroscopy) and Raman spectroscopy is carried out to identify the formation of PEG micelle encapsulating the FAD. FIG. 5 shows the results of the UV-Visible analysis of the FAD alone, the PEG alone, and the PEG-FAD particle. Using Raman spectroscopy, the signature of the PEG in the PEG-FAD complex is confirmed.

The stability of the particle was monitored by UV-Visible and Raman spectroscopy in PBS at pH 4 and 7 at 37° C.

The results at pH 4 in UV-visible spectroscopy are shown in FIG. 6

In UV spectroscopy, a superimposition of the curves can be observed at pH 7 and an insignificant change at pH 4. It can be concluded that the complex is stable in both media and over time.

These results are confirmed by Raman spectroscopy.

### Example 8

Effects of different particles on the proliferation of HELA cells.

The tests are carried out on HeLa cells (uterine cancer metastasis) on 24-well plates seeded at medium/high density (20000 cells/well) in a complete DMEM cell culture medium (Dulbecco's Modified Eagle Medium with serum).

After 24 hours of stabilisation, the cells are incubated for 48 hours in complete DMEM with different concentrations of the products to be tested. Each treatment is carried out in triplicate and provides an average growth rate for each dose considered.

The viability of the cells is evaluated by the MTT test: staining of living cells with a tetrazolium salt and detection by absorbance.

It is expressed as a percentage of growth compared to a cultured control containing the only medium.

The results show that a better effect is achieved with the Gold-FAD-PEG IN particles obtained by the IN method with a proliferation percentage of 71%.

By absorbance detection using the MTT test, the GI50 growth inhibitions are 0.3 and 0.5 μM respectively for gold-PEG-FAD-IN particles coupled with an HIV-TAT-1 targeting agent and gold-PEG-FAD-IN particles coupled with chitosan.

These results corroborate the fact that particle synthesis by the IN method affects inhibiting the proliferation of HeLa cells.

### Example 9

Effects of different nanoparticles on the proliferation of MCF7 cells.

Growth inhibition of MCF7 (breast cancer) cancer cells is tested on MCF7 cells in culture for 48 hours in contact with the gold-FAD-PEG particle with or without a targeting agent obtained by the IN method. It is compared to the activity of a reference anti-cancer product, staurosporin, by reading the absorbance of the colorimetric test based on the reduction of the tetrazilium compound MTS allowing the quantification of viable cells. (Promega—Cell Titer Proliferation Assay).

It is found that growth inhibition, Gi50, is superior to that of staurosporin with 0.45 μM/L for gold-FAD-PEG, 0.70 μM/L for the gold-FAD-PEG-targeting agent being HIV-TAT-1, and about 40 μM/L for staurosporin.

### Example 10

Effects of different particles on the proliferation of HT22 cells.

HT22 cells (murine brain tumour) are tested with the gold-FAD-PEG particle obtained by the IN method.

The inhibition of cancer cell growth by the gold-FAD-PEG particle obtained by the IN method is compared with the activity of staurosporin after 48 hours of incubation and detection of absorbance by MTS test.

The results show a 50% growth inhibition (GI 50) of 1.60 μM/L for the gold-FAD-PEG particle obtained by the IN method and 2.70 μM/L for staurosporin, which is a better effect for the tested particle than for the positive control.

### Example 11

Photothermal properties of the different nanoparticles.

The photothermal effect of FAD according to the invention is measured by controlling the temperature increase of cells mixed with particles obtained with the IN method according to example 1. The cells are then irradiated with infrared radiation by means of a laser thermal probe. The results are shown in FIG. 4.

It can be observed that only the particles of gold-FAD-PEG and the gold-FAD-PEG-targeting agent being HIV-TAT-1 show a thermal rise of more than 4° C.

### Example 12

In vitro viability test of MIA Paca-2 cells.

MIA Paca-2 cells are human pancreatic cancer cells [American Tissue Cell Culture (ATCC, Manassas, Va., USA)] cultured in DMEM medium. The MTT viability test is performed at a cell density of 0.1×10*6 cells/mL. The tested products are incubated for 2 hours at a concentration of 120 μM/L and in triplicate, with culture control (ref: Blank) (100%) and a positive DMSO control (5%). Under these conditions, cell viability is 34% in the presence of FAD alone, 6% with FAD-PEG.

The results show a very good efficiency of the FAD-PEG particle.

### Example 13

Resistance to hydrolysis in serum over time.

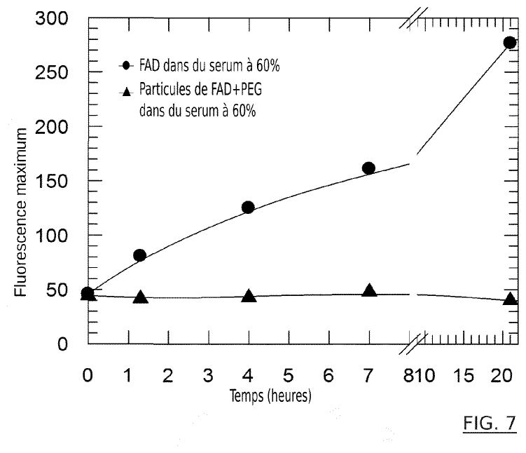

A comparative hydrolysis test of unbound FAD and micrometer-sized FAD-PEG obtained in Example 7 is performed in serum to test resistance to enzymes. It shows the stability of the FAD-PEG particle up to 20 hours when the free FAD is almost completely hydrolyzed. The results are shown in FIG. 7.

### Example 14

Specific features of the compounds according to the invention for tumour cells.

The cytotoxicity of the compounds according to the invention has been tested against tumour cells in CEM (myeloid leukaemia) type lineage and compared to their toxicity towards non-cancerous cells.

Monocytes isolated from peripheral blood in an individual breast (PBMC) seeded 24 hours previously (1×106/mL) and cancer cells of myeloid leukaemia type CEM cells (1×106 cells/mL) were cultured in vitro in the presence or absence of FAD, FAD-PEG, at different doses. 48 hours after incubation, the viability of the cells was evaluated in all cultures by different methods (5×105 cells/mL) cells: exclusion of trypan blue in Malassez cells or incorporation of resazurin and fluorescence analysis compared to a calibration range.

The results show that FAD, FAD-PEG, do not significantly alter the viability of primary non-cancerous cells (IC50>100 mM), and significantly decrease the viability of cancer cells in the lineage (mean IC50=1.6 mM)—

Comparable results (1.1<IC50<1.8 μM) were obtained for FAD on HT-1080 fibrosarcoma cells, HepG2 hepatocellular carcinoma, Huh-7replicon, and MiaPaCA2 pancreatic cancer (IC50=10 μM).

Similarly, in these pancreatic cancer cells, the viability of the cells is significantly altered by FAD (90 μM) this time after only 24 hours of exposure, and FAD-PEG, like the Au-FAD-PEG complex, is extremely effective, (IC50<1 mM), of the same order as DMSO in limiting the survival of cancer cells.

The viability of HT22 neuronal cancer cells (IC50=1.596 mM) is surprisingly more severely impaired by FAD than by Staurosporin (IC50=2.736 mM), another anticancer agent used as a positive control, and follows a different curve depending on whether FAD or Staurosporin is used, suggesting a different mechanism of action of the two anticancer agents.

### Example 15

Intercalation of the molecules according to the invention in the DNA.

Small molecules with aromatic structures are intercalated into DNA, which is the case of some extremely powerful and effective drugs. The hypothesis that FAD intercalates into DNA has been tested using a DNA aptamer grafted onto the surface of nanoparticles (NP).

The aptamer used is a single-stranded, thiolated DNA sequence of formula 5′HS-TT TTT TTT TTT TTT TTC TTC TCT AGC TGA ATA ACC GGA AGT AAC TCA TCG TTT CGA TGA GTT ACT TCC GGT TAT TCA GCT AGA GAA G 3′, grafted to the surface of gold nanoparticles in the presence of a catalyst agent (N-[3-(dimethylamino) propyl]-N′-ethylcarbodiimide/N-hydroxysuccinimide, Sigma-Aldrich) (EDC/NHS), 50 μL, of DNA with a ratio (40 mg/10 mg) to 5 ml of gold NP solution, to allow activation of the carboxylic group (PEG-COOH) on the surface of the nanoparticles for immobilisation of the aptamer by covalent bonds. The bond is made between the carboxyl group of the EDC/NHS-activated PEG and the NH2 groups of the aptamer.

The grafting of the DNA on the surface of the gold nanoparticles is verified by UV spectroscopy (UV-visible absorbance measurements).

The particles were then placed in the presence of FAD or FAG-PEG (5 mg/ml, two formulations) and DNA intercalation, detected and measured by two techniques: UV visible and by RAMAN spectroscopy.

For both FAD and FAD-PEG, the results obtained by both techniques show for the first time that FAD, like FAD-PEG, intercalates to DNA and that the complex formed is stable for at least 24 hours.

These results also suggest at least two types of interaction between FAD and DNA.

The mechanism(s) by which FAD alters the survival of cancer cells selectively are unexpected. Thus, a bond is established between FAD and DNA between alloxazine and adenine and from adenine to a thymidine on the opposite strand. Adenine can be disrupted, while free flavin can behave as an allosteric effector and participate in enzymatic reactions, including activation of ubiquitin.

The activity of the products and molecules according to the invention, as the only active principle selectively targeting cancer cells was then tested in vivo on several cancer models, including hepatocarcinoma, breast cancer, and bladder cancer.

### Pre-Clinical Trials

### Example 16

Evaluation of the anti-tumor activity of FAD and FAD-PEG compounds in vivo.

Breast Cancer Model

Murine cell lines 4T1 (mammary glands),

Culture medium: MEM+FBS (10%)+L-glutamine (2 mM). The cells are cultured in an incubator at 37° C. and 5% CO2.

Installation

Cells of the murine breast cancer cell line 4T1 are subcutaneously implanted in Balb/c mice.

The experimental animals, immunodeficient BALB/c mice (females, 18-22 g, 5 weeks old), are kept in the SPF Laboratory Animal Center at Guangzhong Medical University at a circadian rhythm of 12 h. The mice have free access to food and water.

### Treatments

5-FU is used as a positive control: 5-fluorouracil (10 mg/kg), positive control group, n=10, purity 99%, Sigma Chemical Co., St. Louis, Mo., USA), every 3 days for 3 weeks. Tested products:

NP1: FAD solution (Alpha Aesar, FAD disodium salt hydrate, CAS: 84366-81-4, 94%), at 1 mg/ml.

NP2: FAD/FAD PEG solution, at a dose of 1 mg/ml of total FAD in NaCl 0.9%.

Breast cancer cells from 4T1 mice were resuspended in a 0.9% NaCl solution at a density of 2×107 cells/ml and inoculated (100 μL) into the 2nd pair of left breast pads. From the 7th day after implantation, the tumour is measured (calliper) and the volume calculated using the formula: (length×width×thickness) when the tumour, followed on a sample by scanner imaging, reaches 150-200 mm3 (about J 10).

### The Administration Method

The prophylactic treatment (before injection of the tumour) According to the experimental groups, the products (NP1 FAD alone and NP2 FAD-PEG) is administered in the form of two injections of 100 μl intravenous per day, three days apart, one week before the inoculation of the cancer cells.

Then, in the post-tumoral phase and for all groups, the drugs are injected twice a day (morning and evening) every 3 days for 3 weeks. Blood samples are regularly taken (on days 0, 8, 15, and 22 after tumour injection), the evolution of the tumours in each group is also monitored by scanner imaging. At the end of the experiment, the animals are euthanised using an excess of ketamine/xylazine anaesthetic. The tumors are excised, weighed, and their weight compared to that of negative control to calculate the percentage of growth inhibition of each compound. The liver, kidneys, spleen and thymus are dissected and their ratio to body weight (index) is calculated.

### Results

The volume of the mammary tumour was significantly reduced by more than 16%, and up to more than 25% in the groups of mice treated with FAD alone or FAD-PEG intravenously significantly (p<0.001 in all cases, compared to the control, saline, and/or PEG groups). The thymus index was also increased in mice with decreased tumour volume.

At the same time, biochemical indicators i.e. levels of IL-1α, IL-12P70, TNF α, IL-1 β, and IL-6 are significantly reduced and the IL-10 content is significantly increased in mice treated with FAD alone or FAD-PEG compared to the control groups.

These results indicate that FAD has an anti-cancer effect in a breast cancer model and reduces cancer-related biochemical abnormalities.

### Liver Cancer Model

Mouse HEP 1-6 liver cancer cells, resuspended in a 0.9% NaCl solution, are mixed with a basal matrix of mammalian cells such as Matrigel (a gelatinous protein mixture that allows HEP1-6 cells to grow) 1:1. Each mouse is injected with 1.2×106 cells subcutaneously into the armpit.

The following groups were formed:

Preventive administration:

1. Sham-operated group (control group, n=10);

2. PEG group (solvent control, n=10);

3. Group for subcutaneous injection of NP1 solution (experimental group, n=10);

4. Group for subcutaneous injection of NP2 solution (experimental group, n=10);

5. 5-fluorouracil group (5 mg/kg/day, i.p., positive control group, n=10, 99% purity, Sigma Chemical Co., St. Louis, Mo., USA).

Curative administration:

1. Shame operated group (blank control group, n=10);

2. PEG group (solvent control, n=10);

3. Group for subcutaneous injection of NP1 solution (experimental group, n=10);

4. Group for subcutaneous injection of NP2 solution (experimental group, n=10);

5. Subcutaneous group of 5-fluorouracil solution+NP1 (n=10);

6. Subcutaneous group of 5-fluorouracil solution+NP2 (n=10);

7. 5-fluorouracil group (5 mg/kg/day, i.p., positive control group, n=10, 99% purity, Sigma Chemical Co., St. Louis, Mo., USA).

### Administration Protocols

### Pre-Tumoral Administration:

NP1 or NP2 solution (1 or 5 mg/mL FAD) is injected subcutaneously 2/4/6 days before injection of HEP1-6 cells in mice in a volume of 100 μl once daily. The mice are then inoculated with HEP 1-6 cells, after one week the drug (or placebo) is injected subcutaneously every 2 days for 2 weeks and the animals are anaesthetised with ketamine/xylazine at the end of the experiment. Excessive anaesthesia euthanizes the animals, the tumors are excised and weighed, and the percentage of drug-related inhibition calculated. The liver, kidneys, spleen, and thymus are dissected and their ratio to body weight (index) calculated. After one week of injection, blood is drawn from the eyelids for biochemical indicators.

Method 2 (post-tumoral administration only): After successful tumour modeling in mice, the subcutaneous injection group of NP1 solution and the subcutaneous injection group of NP2 solution are injected with 100 μL of NP1 or NP2 drugs once a day (at 1 or 5 mg/mL), on the two days following the administration of 5-FU. 5-FU (5 mg/mL) is administered continuously for 3 weeks, every 3 days.

In the 5-fluorouracil+NP1 or 5-fluorouracil+NP2 administration groups, the mice received a 100 μL injection of NP1 or NP2 24 hours and 48 hours after the intraperitoneal injection of 5-FU, and are treated once a day for the two days between injections. 5-FU is given once every three days for 3 weeks. At the end of the experiment, the animals are anaesthetised with ketamine/xylazine. After the blood has been drawn, the mice are euthanised with an excess of anaesthetic, the tumour is excised and weighed, and the percentage of drug inhibition calculated. The spleen and thymus are dissected and their ratio to body weight (spleen index and thymus index) calculated. After one week of injection, blood is drawn from the eyelids for biochemical indicators.

The results show that FAD alone has an antitumour effect in the liver cancer model (24% reduction in the volume of liver tumours), on the number of tumours, and that the combination of 5FU and FAD reinforces (potentiates) the antitumour activity of 5-FU (65% reduction in tumour volume) compared to 5-FU alone, FAD alone or FAD PEG alone.

Besides, biological data show that the NP2+5FU/5FU combination, after 30 days of treatment modifies the cytokine level in the blood; IL-1 alpha is decreased compared to controls, IL-10 is increased compared to controls, while IFN gamma, TNF-alpha IL-6, GMCSF are not significantly modified.

There is also a significant increase in all white lines, lymphocytes, monocytes, neutrophils in the FAD or FAD-PEG group (especially for the number of monocytes), and potentiation for the 5FU-FAD-PEG group compared to all the other groups.

The results obtained in all the cancer models tested show that FAD alone has anti-cancer effects on tumour growth and/or metastatic activity. In vivo, FAD and FAD PEG are active and potentiate the effect of another drug, 5FU. In combination with a classical anti-cancer treatment (chemotherapy) such as 5FU, FAD allows a clear improvement (potentiation) of the classical treatment and fewer side effects measured by the volume of the spleen and thymus or by abnormalities of biochemical indicators.

What is claimed is:

1. Flavine adenine dinucleotide (FAD) for use in preventing and/or treating cancer

2. FAD for its use according to claim 1 as the main active ingredient.

3. FAD for its use according to claim 1 as an adjuvant or neoadjuvant to cancer treatment.

4. FAD for its use according to any of the foregoing claims wherein the cancer is selected from the group consisting of breast cancer, prostate cancer, lung cancer, airway, upper and/or lower digestive tract cancer, organs of digestion cancer, kidney cancer, urinary tract cancer, genital organs cancer, skin cancer, ENT sphere cancer, and lymphatic organs cancer.

5. A Composition comprising FAD according to any of the foregoing claims in a therapeutically effective amount and a pharmaceutically acceptable carrier.

6. Composition according to the preceding claim for use in the prevention and/or treatment of cancer comprising a particle comprising a carrier and FAD at least partially encapsulated by the carrier.

7. Composition for its use according to the preceding claim in which the vector is chosen from at least one of metal nanoparticles including gold nanoparticles, biopolymers including Poly Ethylene Glycol (PEG), chitosan, collagen, glucose.

8. Composition for use according to any of claims 5 to 7 wherein the particle is a nanoparticle or a microparticle.

9. Composition for use according to any one of claims 5 to 8 wherein the FAD is bonded to a biopolymer and a gold nanoparticle.

10. Composition for use according to the preceding claim wherein the FAD is covalently bonded to PEG encapsulating at least one gold atom.

11. Composition for use according to any of claims 5 to 9 in which the FAD is bonded to gold atoms by coordination bonding and bonded to the PEG by covalent bonding.

12. Composition according to any one of claims 5 to 8 wherein the FAD is at least partially encapsulated by at least one biopolymer preferably selected from PEG, chitosan, glucose.

13. Composition according to any one of claims 5 to 12 wherein the therapeutically effective amount of FAD comprises an amount of unbound FAD and an amount of FAD associated with a vector.

14. Composition according to any of claims 5 to 13 in a form suitable for parenteral administration including intravenous, intramuscular and subcutaneous, vaginal or rectal administration.

15. Composition according to any of claims 5 to 13 in a form suitable for intra-vesical, or intra-urethral administration.
